(19) United States
(12) Patent Application Publication
(10) Pub. No.: US 2014/0161779 A1
(43) Pub. Date: Jun. 12, 2014

(54) METHODS FOR CONTROLLING PEST

(71) Applicants: Beijing Dabeinong Technology Group Co., Ltd., Beijing (CN); Beijing Green Agrosino Plant Protection Technology Co., Ltd., Beijing (CN); Beijing Dabeinong Technology Group Co., Ltd., Biotech Center, (unknown)

(72) Inventors: Chao HAN, Beijing (CN); Jie Pang, Beijing (CN); Yuejing Kang, Beijing (CN); Haili Liu, Beijing (CN); Yunzhu Zhang, Beijing (CN); Chengwei Zhang, Beijing (CN); Chunping Xu, Beijing (CN); Mei Wei, Beijing (CN); Jincun Huang, Beijing (CN); Kangle Tian, Beijing (CN); Qianqin Wang, Beijing (CN)

(73) Assignees: Beijing Dabeinong Technology Group Co., Ltd., Beijing (CN); Beijing Green Agrosino Plant Protection Technology Co., Ltd., Beijing (CN); Beijing Dabeinong Technology Group Co., Ltd., Biotech Center, Beijing (CN)

(21) Appl. No.: 14/102,705

(22) Filed: Dec. 11, 2013

(30) Foreign Application Priority Data
Dec. 11, 2012 (CN) 201210533580.1

(51) Int. Cl. A01N 63/02; C12N 15/82

(52) U.S. Cl. CPC A01N 63/02; C12N 15/8286
USPC 424/93.21; 514/4.5

(57) ABSTRACT

Involved is a method for controlling the pest Sesamia inferens, comprising a step of contacting Sesamia inferens with Cry1F protein. Sesamia inferens is controlled by the Cry1F protein having pesticidal activity against Sesamia inferens, which is produced in the plants. Compared with the agricultural control, chemical control and biology control currently used in prior art, the present invention can protect the whole plant during whole growth period from the harm of Sesamia inferen. Furthermore, it causes no pollution and no residue and provides a stable and thorough control effect. Also it is simple, convenient and economic.

### CROSS-REFERENCE TO RELATED APPLICATIONS

This application claims priority of Chinese Application No. 201210533580.1 filed on Dec. 11, 2012, the contents of which are incorporated herein in their entireties for all purposes.

### TECHNICAL FIELD

The present invention relates to a method for controlling pest, in particular, a method for controlling pest Sesamia inferens by Cry1F protein expressed in a plant.

### BACKGROUND OF THE INVENTION

Sesamia inferens belongs to Lepidoptera, Noctuidae, which is a polyphagous pest. Besides corn, it also attacks many other graminaceous crops such as rice, sugarcane, broomcorn and the like. This pest widely distributes in the central and southeast China, especially in the most rice-planting area of the south of Shaanxi province and Henan Province. Larva of Sesamia inferens bores into the stem of the crops and hollows it out or even results in the death of the whole plant. The borer holes caused by Sesamia inferens are usually big with a mass of fecula defecated out of the stem. It turns up seriously in low-lying land and the corn fields intercropped with wheat and summer corn is affected more seriously than spring corn.

Corn and sorghum are important food crops in China. Sesamia inferens causes tremendous grain loss every year. It even affects the living conditions of the local populations. At present, agricultural control, chemical control and biological control are usually applied to control Sesamia inferens.

Agricultural control is a method to comprehensively manage multiple factors of the whole farmland ecological system. By means of the regulation of crops, pests and the environmental factors, a farmland ecological environment is created, which is conducive to the crop growth and nonadvantagous to the outbreaking of Sesamia inferens. Treatment of overwinter hosts of Sesamia inferens, reform of the farming system, planting of Sesamia inferens-resistant crops, application of trap crops and intercropping and the like are the main measures to reduce the harm of Sesamia inferens. Because the demands of crop distribution and yield must be guaranteed, the application of agriculture control is limited and cannot serve as an emergency measures. It doesn't work when Sesamia inferens outbreaks.

Chemical control, i.e. pesticides control, is a method to kill pests by using chemical pesticides. Chemical control is an important part of the comprehensive treatment of Sesamia inferens. It is rapid, convenient, simple and economically. Chemical control is an indispensable measure for emergency when Sesamia inferens outbreak. Sesamia inferens can be eliminated before it causes harm and losses by using chemical control. Current chemical control methods mainly include drug granules, spreading of poisoned soil, spraying of medical solution, fumigation of the overwintering adults in straw stacks, etc. But chemical control also has its limitations. For example, the improper operation can usually cause crop phytotoxicity, and pest resistance to drugs. In addition, natural enemies can also be killed by pesticide. Chemical pesticides cause the environmental pollution and destruct the farmland ecosystem as well. Furthermore, pesticide residues may pose a threat to the safety of people and animals and leads to other serious results.

Biological control is a method to control pest populations by using some beneficial organisms or biological metabolites, which finally reduces or eliminates pests. Biological control is safe to human and livestock and causes less pollution to the environment. And some pests can be controlled in long-term by using biological control. But the control effect is usually instable, and the investment cannot be coordinated according to the different occurrences of Sesamia inferens attack.

In order to solve the limitations of the agricultural control, chemical control and biological control in practical application, the scientists found that, by means of transfecting genes encoding pesticidal protein into plants, some insect-resistant transgenic plants were obtained to control pests. Cry1F pesticidal protein is one of the numerous pesticidal proteins, which is an insoluble parasporal crystal protein produced by Bacillus thuringiensis.

Cry1F protein is taken in by insects and enters into their midgut and this toxic protein protoxin is dissolved in the insect midgut under an alkaline condition. N- and C-ends of the protein are digested by an alkaline protease and this protoxin turns to active fragments. These active fragments bind with the receptors on the epithelial cell membrane of the insect midgut and insert into the cell membrane, which causes cell membrane perforation lesions. It damages the osmotic pressure and pH balances inside and outside of the cell membrane, disrupts the digestion process and eventually result in the death of the insect.

It has been proved that Cry1F transgenic plants can resist Lepidoptera pests such as Agrotis ypsilon Rottemberg. However, so far there is no report about the application of transgenic plants expressing Cry1F protein to control Sesamia inferens.

### SUMMARY OF THE INVENTION

The present invention is to provide a method for controlling the pests. It is the first time to control Sesamia inferens by producing transgenic plants expressing Cry1F protein. The present invention effectively overcomes the technical limitations of the prior art such as agricultural control, chemical control and biological control.

In one aspect, the present invention provides a method for controlling Sesamia inferens comprising a step of contacting Sesamia inferens with Cry1F protein.

In some embodiments, the Cry1F protein is Cry1Fa protein.

In some embodiments, the Cry1Fa protein is present in a plant cell that can express the Cry1Fa protein, and said Sesamia inferens contacts with the Cry1Fa by ingestion of the cell.

In some embodiments, the Cry1Fa protein is present in a transgenic plant that expresses the Cry1Fa protein, and Sesamia inferens contacts with the Cry1Fa protein by ingestion of a tissue of the transgenic plant such that the growth of Sesamia inferens is suppressed or even resulting in the death of Sesamia inferens to achieve the control of the damage caused by Sesamia inferens.

In some embodiments, the transgenic plant is in any growth period.

In some embodiments, the tissue of the transgenic plants is selected from the group consisting of lamina, stalk, tassel, ear, anther and filament.

In some embodiments, the control of the damage caused by Sesamia inferens is independent of the planting location.

In some embodiments, the control of the damage caused by Sesamia inferens is independent of the planting time.

In some embodiments, the plant is selected from the group consisting of corn, rice, sorghum, wheat, millet, cotton, reed, sugarcane, water bamboo, broad bean and rape.

In some embodiments, prior to the step of contacting, a step of growing a plant which contains a polynucleotide encoding the Cry1Fa protein is performed.

In some embodiments, the amino acid sequence of the Cry1Fa protein comprises the amino acid sequence of SEQ ID NO: 1 or SEQ ID NO: 2. The nucleotide sequence encoding Cry1Fa protein comprises a nucleotide sequence of SEQ ID NO: 3 or SEQ ID NO: 4.

Based on above technical solutions, the plant further contains at least a second nucleotide sequence, which is different from that encoding the Cry1Fa protein.

In some embodiments, the second nucleotide encodes a Cry-like pesticidal protein, Vip-like pesticidal protein, a protease inhibitor, lectin, α-amylase or peroxidase.

In some embodiments, the second nucleotide encodes a Cry1Ab protein, a Cry1Ac protein, Cry1Ba protein or Vip3A protein.

In some embodiments, the second nucleotide comprises a nucleotide sequence of SEQ ID NO: 5 or SEQ ID NO: 6.

Optionally, the second nucleotide is dsRNA which inhibits the important gene(s) of a target pest.

In present invention, Cry1F protein is expressed in a transgenic plant accompanied by the expressions of one or more Cry-like pesticidal proteins and/or Vip-like pesticidal proteins. This co-expression of more than one kind of pesticidal toxins in a same transgenic plant can be achieved by transfecting and expressing the genes of interest in plants by genetic engineering. In addition, Cry1F protein can be expressed in one plant (Parent 1) through genetic engineering operations and Cry-like pesticidal protein and/or Vip-like pesticidal proteins can be expressed in the second plant (Parent 2) through genetic engineering operation. The progeny expressing all genes of Parent 1 and Parent 2 can be obtained by crossing Parent 1 and Parent 2.

RNA interference (RNAi) refers to a highly conserved and effective degradation of specific homologous mRNA induced by double-stranded RNA (dsRNA) during evolution. Therefore RNAi technology is applied to specifically knock out or shut down the expression of a specific gene of the target pest in present invention.

Both Sesamia inferens and Agrotis ypsilon Rottemberg belong to Lepidoptera, Noctuidae. Both of them are polyphagous pests but obviously appetite plants of gramineae. Usually they mostly harm corn, rice, sorghum, sugarcane and so on. In spite of this, Sesamia inferens and Agrotis ypsilon Rottemberg are two definitely and completely different species in biology. The major differences between them are shown as below:

1. Distribution areas are different. Sesamia inferens widely distributes in the central and southeast of China, especially in the most rice-planting area of the south of Shaanxi province and Henan Province and corn-planting area of the southwest of China. Besides China, Sesamia inferens also distributes in the Southeast Asian countries planting rice, corn and sugarcane, including Vietnam, Laos, India, etc. While Agrotis ypsilon Rottemberg is a worldwide pest as well as in China, especially much distributes in the humid areas with rich rainfall, such as Yangtze River basin and South-East coastal areas of China. Agrotis ypsilon Rottemberg also appears in the eastern and southern humid regions of northeastern China.

2. Harmful habits are different. Sesamia inferens belongs to boring pests. Damage caused by it includes, for example the following. Its larva bores into the crop stems, causing dead heart seedlings or the death of the whole plant. The borer holes caused by Sesamia inferens are usually big with a mass of fecula defecated out of the stem which is sandwiched between the leaf sheath and stem. The harmed lamina and leaf sheath turn yellow. Newly hatched Sesamia inferens larvae don't scatter but cluster inner side of the leaf sheath, boring leaf sheath and caulicle. After the 3rd instars, the larvae scatter to neighboring plants and can harm 5-6 strains. This is a seriously harming period of Sesamia inferens. If temperature turns to above 10□ earlier in the early spring, Sesamia inferens occurs earlier. It turns up seriously in low-lying land and the corn fields intercropped with wheat and summer corn is affected more seriously than spring corn. In contrast, Agrotis ypsilon Rottemberg belongs to soil insect. The 1st and 2nd instars larvae can cluster and feed on the young leaves on the top of seedlings day and night; after 3rd instars, the larvae scatter. The larvae move quickly, behave in feigning death and are extremely sensitive to the light. They may shrink conglobately when disturbed. They hide between the wet and dry layers of the surface soil during the daytime and come out of the ground, bite the seedlings and drag them into holes underground or directly bite the unearthing seeds. After the main stem of the seedlings get indurated, they change to eat young leaves, laminae and the growing points. They may migrate when food is not enough or they need to search for wintering sites. Elder larvae harm seedlings with a high shear rate and big appetite.

3. The morphological characteristics are different.

1) Different egg morphology: Sesamia inferen's egg is oblate in shape, with vertical and horizontal thin lines on the surface. The egg is white in color initially, but turns grey yellow with age. They consorte or scatter, and arrange in 2-3 lines usually. In contrast, Agrotis ypsilon Rottemberg's egg is in the shape of a steamed bun. The egg bears ribs that radiate from the apex and it is white in color initially, but turns yellow with age. A black point usually shows on the top of the egg before eclosion.

2) Different larva morphology: Larval body length of Sesamia inferen's is reported to be about 30 mm for the final instar. In appearance, the head capsule is colored ranging from red-brown to dark-brown and the dorsal and back surfaces are light prunosus. There are five to seven instars. But the larva of Agrotis ypsilon Rottemberg is cylindrical in shape and the length of the mature larva ranges from 37 to 50 mm. Head capsule of the larva is colored brown with irregular reticulate of pitchy color. The body is colored ranging from gray-brown to dark-brown. The body surface is rough and covered with numerous dark spots. Dorsal lines, sub-dorsal lines and spiracle lines are pitchy in color. Pronotum is dark brown in color. There are two obvious, dark brown longitudinal strips on the tawny subanal laminae. Pereiopods and abdominal feet are tawny in color.

3) Different Pupa morphology: Pupa of Sesamia inferen is 13-18 mm in length, stout and red-brown. Abdomen is covered with gray powder; apex abdominis has 3 hooked spines. Pupa of Agrotis ypsilon Rottemberg is 18-24 mm in length, russet and bright. Mouthpiece and the wing buds terminal are aligned and both stretch up to the posterior border of the fourth urite. The center of the anterior border of the back from the fourth to the seventh segments is dark brown in color and with thick punctums. Bilateral small punctums extend to the stigma. The anterior border of venter aspect from the fifth to seventh segments also has small punctums and a pair of short apex abdominis is on the abdominal end.

4) Different adult morphology: Female moth of adult Sesamia inferen is 15 mm in length and the wingspan is about 30 mm. The head and thorax are fawn in color and abdomen ranges from light yellow to pale in color. Antennae are filamentous; the forewings are nearly rectangular and light grey-brown in color. Four small black spots are arranged quadrilaterally. Male moth is about 12 mm and the wingspan is 27 mm in length. The antenna is pectinated. The adult Agrotis ypsilon Rottemberg is 17-23 mm and the wingspan is 40-54 mm in length. The head and thorax are dark brown, legs are brown in color. The foreleg tibia and the exterior margin of the tarsus are gray brown. The end of each segment of the midleg and hindleg has grey-brown annulation. Forewings are brown, its anterior border is black brown and the color within the anterior border is dark brown. The baseline is light brown. The double lines of wavy, interior transverse linesare black. Inside of the black annulation is a round grey spot. Kidney shaped lines are black and have a black edge and a wedge-shaped black line in the exterior center stretched out to the exterior transverse line, the middle transverse line is dark brown and the double lines of the wavy, exterior transverse lines are brown. The irregular, serrated, penultimated exterior marginal line is gray and its interior marginal line between the midrib has three tines. There are small black dots on each vein between the penultimated exterior marginal line and the exterior transverse line. The exterior edge line is black, between the exterior transverse line and penultimated exterior marginal line is light brown, and beyond the exterior marginal line is dark brown. Underwing is gray, the longitudinal vein and marginal lines are brown and the back of the abdomen is gray.

4. Growth habit and regularity of outbreak are different. Sesamia inferen appears 2-4 generations a year, decreasing with the increase of altitude and increasing with the temperature rise. For example, 2-3 generations occur on the Yunnan-guizhou plateau per year, 3-4 generations occur in Jiangsu province and Zhejiang province per year, 4 generations occur in Jiangxi province, Hunan province, Hubei province and Sichuan province per year, 4-5 generations occur in Fujian province, Guangxi province and Kaiyuan City of Yunnan province and 6-8 generations occur in the southern of Guangdong province and Taiwan. In temperate zone, the mature larvae overwinter in the parasitic residual bodies (such as the haulms or rhizomes of water bamboo and rice) or in the soil near the ground. In the middle of March of the following year (the temperature above 10° C.) larvae start pupation and start eclosion at 15° C. In the early April they begin to copulate and oviposit and after 3-5 days, the copulation and oviposition reach the fastigium. And the eclosion fastigium happens in late April. Adults hide in the daytime and often perch between plants and in the evening activities begin. Its phototaxis is weak and lifetime is about 5 days. Female moths start to oviposit 2-3 days after copulation and after 3-5 days the oviposition reaches the fastigium. They prefer to oviposit on the maize seedling and the field side. Eggs mainly locate at the inside of leaf sheaths of the second and third segments near the ground of the corn plants of which the haulm is slimmer and the obvolvent of the leaf sheath is not tight, which can account for more than 80% of oviposition amount. Each female can spawn 240 eggs and the oviposition duration of the first generation is 12 days, and that of the second and third generations is 5-6 days. Larval stage of the first generation is about 30 days, the second generation of about 28 days, and the third generation of about 32 days. Pupal stage is of 10-15 days. Female moth flies weakly and oviposition is relatively concentrated. The population density is high and harms heavily in the place close to insect source. The Agrotis ypsilon Rottemberg occurs 3-4 generations per year, the mature larvae or pupae overwinter in the soil. In the early March of spring, adult begins to appear and two fastigiums of eclosion will generally occur between the middle and late march and between the early and middle April. Adult is not active during the day time. From evening until the first half of the night, their activities are the most vigorous. They prefer sour, sweet and winy fermented materials and various nectars. They have phototoxic. Larvae have 6 instars. 1, 2 instar larvae hide in the heart leaves of weeds or crops firstly, feed day and night but eat little so they don't harm significantly. 3 instar larvae hide under top soil during the day time and do harm at night. Appetite of 5, 6 instar larvae increase a lot and each larva can bite off 4-5 seedlings even more than 10 seedlings per night. Resistance to drugs of larvae after 3 instar increases significantly. From the end of March to the middle of April is the serious period of the harm of the first generation larvae. The occurrence and harm can be found from October until April of the following year. 2-3 generations per year in northwest China, 2-3 generations per year in north of the Great Wall, 3 generations per year from south of the Great Wall to the north of the Yellow River, 4 generations per year from the south of the Yellow River to Yangtze River, 4-5 generations per year in the south of the Yangtze river and 6-7 generations per year in tropics of South Asia. However many generations happen per year, the most harmful one is the first generation larva. Overwintering adults occur in February in the South. Maximum eclosion happens from the late March to early and middle April in most regions of China, but in late April in Ningxia province and Inner Mongolia province. Eclosion of adult Agrotis ypsilon Rottemberg usually happens from 3 p.m. to 10 p.m. They hide in the locations such as cracks and sundries during the day time and begin to fly and forage in the evening. After 3-4 days, they begin to copulate and oviposit. Eggs are scattered on low and thickleaf weeds and seedling, a few on dead leaves or in soil seam. Most eggs are near the ground. Each female can spawn 800-1000 eggs, even more than 2000 eggs. The oviposition duration is about 5 days, larva has 6 instars and 7-8 instars individually. Larva periods vary widely from place to place but the first generation is of about 30-40 days. Matured larva pupates in a soil chamber about 5 cm deep, pupal stage is of about 9-19 days. High temperature is nonadvantageous to the development and reproduction of Agrotis ypsilon Rottemberg, and thus it rarely happens in summer and the appropriate temperature is of 15° C.-25° C. Winter temperature is too low so that larval mortality of Agrotis ypsilon Rottemberg increases in winter. It happens frequently in the low and moist location with abundant rainfall. It is a sign of Agrotis ypsilon Rottemberg's outbreak if it rained much in the autumn of last year and the soil moisture is high and weeds grow heavily, which benefit the oviposition of adults and feed of the larvae. But if rainfall is too much and humidity is too high, it will go against the development of larvae. Early instar larvae easily die after flooding. It harms seriously if water content of the soil is of 15-20% in the fastigium of adults oviposition stage. Sandy loam which is permeable to rapidly drain away water is suitable for the propagation of Agrotis ypsilon Rottemberg; and it happens less in heavy clay soil and sandy soil.

In conclusion, it can be confirmed that Sesamia inferen and Agrotis ypsilon Rottemberg are different pests with far genetic relationship and they can't copulate to get descendants.

The genome of the plants, the plant tissues or the plant cells described in the present invention, refers to any genetic material in the plants, the plant tissues, or the plant cells, including the nucleus, plastids and the genome of mitochondrial.

As described in the present invention, polynucleotides and/or nucleotides form a complete “gene”, encoding proteins or polypeptides in the host cells of interest. It is easy for one skilled in the art to realize that polynucleotides and/or nucleotides in the present invention can be introduced under the control of the regulatory sequences of the target host.

As well known by one skilled in the art, DNA exists typically as double strands, which are complementary with each other. When DNA is replicated in plants, other complementary strands of DNA are also generated. Therefore, the polynucleotides exemplified in the sequence listing and complementary strands thereof are comprised in this invention. The “coding strand” generally used in the art refers to a strand binding with an antisense strand. For protein expression in vivo, one of the DNA strands is typically transcribed into a complementary strand of mRNA, which serves as the template of protein expression. Actually, mRNA is transcribed from the “antisense” strand of DNA. “Sense strand” or “coding strand” contains a series of codons (codon is a triplet of nucleotides that codes for a specific amino acid), which might be read as open reading frames (ORF) corresponding to genes that encode target proteins or peptides. RNA and PNA (peptide nucleic acid) which are functionally equivalent with the exemplified DNA were also contemplated in this invention.

Nucleic acid molecule or fragments thereof were hybridized with the Cry1Fa gene under stringency condition in this invention. Any regular methods of nucleic acid hybridization or amplification can be used to identify the existence of the Cry1Fa gene in present invention. Nucleic acid molecules or fragments thereof are capable of specifically hybridizing with other nucleic acid molecules under certain conditions. In present invention, if two nucleic acid molecules can form an antiparallel nucleic acid structure with double strands, it can be determined that these two molecules can hybridize with each other specifically. If two nucleic acid molecules are completely complementary, one of two molecules is called as the “complement” of another one. In this invention, when every nucleotide of a nucleic acid molecule is complementary with the corresponding nucleotide of another nucleic acid molecule, it is identified the two molecules are “completely complementary”. If two nucleic acid molecules can hybridize with each other so that they can anneal to and bind to each other with enough stability under at least normal “low-stringency” conditions, these two nucleic acids are identified as “minimum complementary”. Similarly, if two nucleic acid molecules can hybridize with each other so that they can anneal to and bind to each other with enough stability under normal “high-stringency” conditions, it is identified that these two nucleic acids are “complementary”. Deviation from “completely complementary” can be allowed, as long as the deviation does not completely prevent the two molecules to form a double-strand structure. A nucleic acid molecule which can be taken as a primer or a probe must have sufficiently complementary sequences to form a stable double-strand structure in the specific solvent at a specific salt concentration.

In this invention, basically homologous sequence refers to a nucleic acid molecule, which can specifically hybridize with the complementary strand of another matched nucleic acid molecule under “high-stringency” condition. The stringency conditions for DNA hybridization are well-known to one skilled in the art, such as treatment with 6.0*sodium chloride/sodium citrate (SSC) solution at about 45° C. and washing with 2.0*SSC at 50° C. For example, the salt concentration in the washing step is selected from 2.0*SSC and 50° C. for the “low-stringency” conditions and 0.2*SSC and 50° C. for the “high-stringency” conditions. In addition, the temperature in the washing step ranges from 22° C. for the “low-stringency” conditions to 65° C. for the “high-stringency” conditions. Both temperature and the salt concentration can vary together or only one of these two variables varies. In some embodiments, the stringency condition used in this invention might be as below. SEQ ID NO: 3 or SEQ ID NO: 4 is specifically hybridized in 6.0*SSC and 0.5% SDS solution at 65° C. Then the membrane was washed one time in 2*SSC and 0.1% SDS solution and 1*SSC and 0.1% SDS solution, respectively.

Therefore, the insect-resistant sequences which can hybridize with SEQ ID NO: 3 and/or SEQ ID NO: 4 under stringency conditions were comprised in this invention. These sequences were at least about 40%-50% homologous or about 60%, 65% or 70% homologous, even at least 75%, 80%, 85%, 90%, 91%, 92%, 93%, 94%, 95%, 96%, 97%, 98%, 99% or higher homologous to the sequences of present invention.

Genes and proteins described in the present invention include not only the specifically exemplified sequences, but also parts and/or fragments (including deletion(s) in and/or at the end of the full-length protein), variants, mutants, substitutes (proteins containing substituted amino acid(s)), chimeras and fusion proteins retaining the pesticidal activity thereof. The “variants” or “variation” refers to the nucleotide sequences encoding the same one protein or encoding an equivalent protein having pesticidal activity. The “equivalent protein” refers to the proteins that have the same or the substantially same bioactivity of anti-Sesamia inferens as that of the claimed proteins.

The “fragment” or “truncation” of the DNA or protein sequences as described in this invention refers to a part or an artificially modified form thereof (e.g., sequences suitable for plant expression) of the original DNA or protein sequences (nucleotides or amino acids) involved in present invention. The sequence length of said sequence is variable, but it is long enough to ensure that the (encoded) protein is an insect toxin.

It is easy to modify genes and to construct genetic mutants by using standard techniques, such as the well-known point mutation technique. Another example method is that described in the U.S. Pat. No. 5,605,793 of randomly splitting DNA and then reassembling them to create other diverse molecules. Commercially available endonucleases can be used to make gene fragments of full-length gene, and exonuclease can also be operated following the standard procedures. For example, enzymes such as Bal31 or site-directed mutagenesis can be used to remove nucleotides systematically from the ends of these genes. Various restriction enzymes can also be applied to obtain genes encoding active fragments. In addition, active fragments of these toxins can be obtained directly using the proteases.

In the present invention, the equivalent proteins and/or genes encoding these proteins could be derived from B.t. isolates and/or DNA libraries. There are many ways to obtain the pesticidal proteins of the invention. For example, the antibodies raised specifically against the pesticidal protein disclosed and protected in present invention can be used to identify and isolate other proteins from protein mixtures. In particular, the antibody may be raised against the most constant part of the protein and the most different part from other B.t. proteins. These antibodies then can be used to specifically identify equivalent proteins with the characteristic activity using methods of immunoprecipitation, enzyme linked immunosorbent assay (ELISA) or Western blotting assay. It is easy to prepare the antibodies against the proteins, equivalent proteins or the protein fragments disclosed in the present invention using standard procedures in this art. The genes encoding these proteins then can be obtained from microorganisms.

Due to redundancy of the genetic codons, a variety of different DNA sequences can encode one same amino acid sequence. It is available for one skilled in the art to achieve substitutive DNA sequences encoding one same or substantially same protein. These different DNA sequences are comprised in this invention. The “substantially same” protein refers to a sequence in which certain amino acids are substituted, deleted, added or inserted but pesticidal activity thereof is not substantially affected, and also includes the fragments remaining the pesticidal activity.

Substitution, deletion or addition of some amino acids in amino acid sequences in this invention is conventional technique in the art. In some embodiments, such an amino acid change includes: minor characteristics change, i.e. substitution of reserved amino acids which does not significantly influence the folding and/or activity of the protein; short deletion, usually a deletion of about 1-30 amino acids; short elongation of amino or carboxyl terminal, such as a methionine residue elongation at amino terminal; short connecting peptide, such as about 20-25 residues in length.

The examples of conservative substitution are the substitutions happening in the following amino acids groups: basic amino acids (such as arginine, lysine and histidine), acidic amino acids (such as glutamic acid and aspartic acid), polar amino acids (e.g., glutamine and asparagine), hydrophobic amino acids (such as leucine, isoleucine, and valine), aromatic amino acids (e.g., phenylalanine, tryptophan and tyrosine), and small molecular amino acids (such as glycine, alanine, serine and threonine and methionine). Amino acid substitutions generally not changing specific activity are well known in the art and have been already described in, for example, “Protein” edited by N. Neurath and R. L. Hill, published by Academic Press, New York in 1979. The most common substitutions are Ala/Ser, Val/Ile, Asp/Glu, Thr/Ser, Ala/Thr, Ser/Asn, Ala/Val, Ser/Gly, Tyr/Phe, Ala/Pro, Lys/Arg, Asp/Asn, Leu/Ile, Leu/Val, Ala/Glu and Asp/Gly, and reverse substitutions thereof.

Obviously, for one skilled in the art, such a substitution may happen outside of the regions which are important to the molecular function and still cause the production of active polypeptides. For the polypeptide of the present invention, the amino acid residues which are required for their activity and chosen as the unsubstituted residues can be identified according to the known methods of the art, such as site-directed mutagenesis or alanine-scanning mutagenesis (see, e.g. Cunningham and Wells, 1989, Science 244:1081-1085). The latter technique is carried out by introducing mutations in every positively charged residue in the molecule and detecting the insect-resistant activity of the obtained mutation molecules so as to identify the amino acid residues which are important to the activity of the molecules. Enzyme-substrates interaction sites can also be determined by analyzing its three-dimensional structure, which can be determined through some techniques such as nuclear magnetic resonance (NMR) analysis, crystallography, or photoaffinity labeling (see, for example, de Vos et al., 1992, Science 255:306-312,; Smith, et al., 1992, J. Mol. Biol 224:899-904; Wlodaver et al., 1992, FEBS Letters 309:59-64).

In the invention, Cry1F protein includes but is not limited to Cry1Fa2, Cry1Fa3, Cry1Fb3, Cry1Fb6 or Cry1Fb7 protein, or the pesticidal fragments or functional domains with pesticidal activity against Sesamia inferen, whose amino acid sequences are at least 70% homologous with that of the protein mentioned above.

Therefore, amino acid sequences which have certain homology with the amino acid sequences set forth in SEQ ID NO. 1 and/or SEQ ID No. 2 are also comprised in this invention. The sequence similarity/homology between these sequences and the sequences described in the present invention are typically more than 60%, preferably more than 75%, more preferably more than 80%, even more preferably more than 90% and more preferably more than 95%. The preferred polynucleotides and proteins in the present invention can also be defined according to more specific ranges of the homology and/or similarity. For example, they have a homology and/or similarity of 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% with the sequences described in this invention.

Regulatory sequences described in this invention include but are not limited to a promoter, transit peptide, terminator, enhancer, leading sequence, introns and other regulatory sequences that can be operably linked to the Cry1F protein.

The promoter is a promoter expressible in plants, wherein said “a promoter expressible in plants” refers to a promoter which ensures that the coding sequences bound with the promoter can be expressed in plant cells. The promoter expressible in plants can be a constitutive promoter. The examples of promoters capable of directing the constitutive expression in plants include but are not limited to 35S promoter derived from Cauliflower mosaic virus, Ubi promoter, promoter of GOS2 gene derived from rice and the like. Alternatively, the promoter expressible in plants can be a tissue-specific promoter, which means that the expression level directed by this promoter in some plant tissues such as in chlorenchyma, is higher than that in other tissues of the plant (can be measured through the conventional RNA test), such as the PEP carboxylase promoter. Alternatively, the promoter expressible in plants can be wound-inducible promoters as well. Wound-inducible promoters or promoters that direct wound-inducible expression manners refer to the promoters by which the expression level of the coding sequences can be increased remarkably compared with those under the normal growth conditions when the plants are subjected to mechanical wound or wound caused by the gnaw of an insect. The examples of wound-inducible promoters include but are not limited to the promoters of genes of protease inhibitor of potato and tomato (pin I and pin II) and the promoter of maize protease inhibitor gene (MPI).

The transit peptide (also called secretary signal sequence or leader sequence) directs the gene products into specific organelles or cellular compartment. For the receptor protein, the transit peptide can be heterogeneous. For example, sequences encoding chloroplast transit peptide are used to lead to chloroplast; or ‘KDEL’ reserved sequence is used to lead to the endoplasmic reticulum or CTPP of the barley lectin gene is used to lead to the vacuole.

The leader sequences include but are not limited to small RNA virus leader sequences, such as EMCV leader sequence (encephalomyocarditis virus 5′ non coding region); Potato virus Y leader sequences, such as MDMV (maize dwarf Mosaic virus) leader sequence; human immunoglobulin heavy chain binding protein (BiP); untranslated leader sequence of the coat protein mRNA of Alfalfa Mosaic virus (AMV RNA4); Tobacco Mosaic virus (TMV) leader sequence.

The enhancer includes but is not limited to Cauliflower Mosaic virus (CaMV) enhancer, Figwort mosaic virus (FMV) enhancer, carnations etched ring virus (CERV) enhancer, cassava vein Mosaic virus (CsVMV) enhancer, mirabilis mosaic virus (MMV) enhancer, Cestrum yellow leaf curling virus (CmYLCV) enhancer, Cotton leaf curl Multan virus (CLCuMV), Commelina yellow mottle virus (CoYMV) and peanut chlorotic streak caulimovirus (PCLSV) enhancer.

For the application of monocotyledon, the introns include but are limited to maize hsp70 introns, maize ubiquitin introns, Adh intron 1, sucrose synthase introns or rice Act1 introns. For the application of dicotyledonous plants, the introns include but are not limited to CAT-1 introns, pKANNIBAL introns, PIV2 introns and “super ubiquitin” introns.

The terminators can be the proper polyadenylation signal sequences playing a role in plants. They include but are not limited to polyadenylation signal sequence derived from Agrobacterium turnefaciens nopaline synthetase (NOS) gene, polyadenylation signal sequence derived from protease inhibitor II (pin II) gene, polyadenylation signal sequence derived from peas ssRUBISCO E9 gene and polyadenylation signal sequence derived from α-tubulin gene.

The term “operably linked” described in this invention refers to the linking of nucleic acid sequences, which provides the sequences the required function of the linked sequences. The term “operably linked” described in this invention can be to link a promoter with the sequences of interest, which makes the transcription of these sequences under the control and regulation of the promoter. When the sequence of interest encodes a protein and the expression of this protein is required, the term “operably linked” indicates that the linking of the promoter and said sequence makes the obtained transcript to be effectively translated. If the linking of the promoter and the coding sequence results in transcription fusion and the expression of the encoding protein are required, such a linking is generated to make sure that the first translation initiation codon of the obtained transcript is the initiation codon of the coding sequence. Alternatively, if the linking of the promoter and the coding sequence results in translation fusion and the expression of the encoding protein is required, such a linking is generated to make sure that the first translation initiation codon of the 5′ untranslated sequence is linked with the promoter, and such a linking way makes the relationship between the obtained translation products and the open reading frame encoding the protein of interest meet the reading frame. Nucleic acid sequences that can be operably linked include but are not limited to sequences providing the function of gene expression (i.e. gene expression elements, such as a promoter, 5′ untranslated region, introns, protein-coding region, 3′ untranslated region, polyadenylation sites and/or transcription terminators); sequences providing the function of DNA transfer and/or integration (i.e., T-DNA boundary sequences, recognition sites of site-specific recombinant enzyme, integrase recognition sites); sequences providing selectable function (i.e., antibiotic resistance markers, biosynthetic genes); sequences providing the function of scoring markers; sequences assistant with the operation of sequences in vitro or in vivo (polylinker sequences, site-specific recombinant sequences) and sequences providing replication function (i.e. origins of replication of bacteria, autonomously replicating sequences, centromeric sequences).

The term “pesticidal” described in this invention means it is poisonous to crop pests. More specifically, the target insects are Sesamia inferen Walker pests.

Cry1F protein of this invention is poisonous to Sesamia inferen Walker pests. The plants mentioned in the invention, especially the sorghum and maize, contain exogenous DNA in their genome. The exogenous DNA contains nucleotide sequences encoding Cry1F protein. When Sesamia inferens contacts with the Cry1Fa protein through feeding with the tissues of these transgenic plants, growth of Sesamia inferens is inhibited and the death of Sesamia inferens is caused eventually. The term “inhibition” refers to lethal or sub-lethal. At the same time, the plants should be normal in morphology, and can be cultivated with the normal means for the consumption and/or generation of products. In addition, the requirement of chemical or biological pesticides of the plant can be essentially eliminated (the chemical or biological pesticides are the ones against Sesamia inferen targeted by Cry1F protein).

The expression level of pesticidal crystal proteins (ICP) in the plant materials can be determined using various methods described in this field, such as the method of quantifying mRNA encoding the pesticidal protein in the tissue through using specific primers, or the method of quantifying the pesticidal protein directly and specifically.

The pesticidal effect of ICP in the plants can be detected by using different tests. The target insects of the present invention are mainly Sesamia inferen.

The Cry1F protein in the present invention can have the amino acid sequences set forth in SEQ ID NO: 1 and/or SEQ ID NO: 2 in the sequence listing. The protein contains not only coding region of Cry1F protein but also other elements, such as regions which encode selectable marker proteins.

In addition, the expression cassettes containing the nucleotide sequence coding the Cry1F protein of present invention can also be co-expressed with at least one kind of proteins encoded by herbicide-resistance genes in plants, resulting that the transgenic plants obtained have both high pesticidal activity and herbicide-resistance activity. The herbicide-resistance genes include but are not limited to glufosinate-resistance genes (such as bar gene and pat gene), phenmedipham-resistance genes (such as pmph gene), glyphosate-resistance genes (such as EPSPS gene), bromoxynil-resistance genes, sulfonylurea-resistance genes, dalapon-resistance genes, genes resistant to cyanamide or genes resistant to glutamine synthetase inhibitors (such as PPT).

In this invention, exogenous DNA was introduced into plants. For example, genes, expression cassettes or recombinant vectors encoding Cry1F protein were introduced into plant cells. The conventional transformation methods include but are not limited to Agrobacterium-mediated transfection, Particle Bombardment, direct intake of DNA into protoplast, electroporation or silicon-mediated DNA introduction.

The present invention provides a method of controlling the pests with the following advantages:

1. The internal cause-based control. The prior arts are mainly to control the harm of Sesamia inferen pests by external action (i.e. external cause), such as agricultural control, chemical control and biological control; while the invention is to control Sesamia inferen pests through Cry1F protein produced in the plants which is capable of killing Sesamia inferen pests.

2. No pollution and no drug residue. Although the chemical control used in prior art has played a role in the controlling of Sesamia inferen, it also caused pollution, destruction and drug residues and to human, livestocks and the farmland ecosystem; through using the method of controlling Sesamia inferen pests, these bad consequences can be eliminated.

3. Controlling in the whole growth periods. Each of the methods of controlling the Sesamia inferen pests employed in prior art is staged, while the method of present invention is capable of protecting plants during their whole growth period. Transgenic plants (Cry1F protein) can avoid from the harm of Sesamia inferen from germination, growth, until blossom and fruit production.

4. The whole plant control. Most methods of controlling the Sesamia inferen pests of prior art are localized, such as leaf surface spraying. While this invention is to protect the whole plants from Sesamia inferen, such as leaf, stem, tassel, ear, anther and filament of the transgenic plant (Cry1F protein).

5. The stable effects. Biological pesticides used in prior art are sprayed directly to the crop surface, resulting the degradation of the actively crystallized proteins (including Cry1F protein) in the environment. Compared with this, Cry1F protein mentioned in the present invention is expressed in the plant, thereby effectively avoiding the deficiency of instability of the biological pesticides in nature. Furthermore, control effects of the transgenic plants (Cry1F protein) of this invention are stable and consistent in different locations, time and genetic backgrounds.

6. It is simple, convenient and economic. Biological pesticides used in prior art are susceptible to be degraded in the environment, and therefore repeated production and application are required, which bring practical difficulties on agricultural production and thus greatly increase the cost. The only thing required for this the invention is to plant transgenic plants expressing Cry1F protein, without the need of other measures, so that plenty of manpower, material and financial resources are saved.

7. The complete effect. The control effect of existing methods to control Sesamia inferen pests is incomplete and can only bring out an alleviation effect. Compared with this, the transgenic plants (Cry1F protein) of this invention can result a massive death of the newly hatched larvae of Sesamia inferen. Furthermore, it can also greatly inhibit the development progress of the rarely survival larva. After 3 days, larvae still remain in the early hatched status or in the status between early hatched status and negative control status, which are obviously maldeveloped, and the development thereof has stopped. However transgenic plants are generally slightly harmed.

The technical solutions of this invention will be further described through the appended figures and examples as following.

### BRIEF DESCRIPTION OF THE DRAWINGS

FIG. 1 shows the scheme to construct the recombinant cloning vector DBN01-T containing Cry1Fa-01 nucleotide sequence for pest control in this invention;

FIG. 2 shows the scheme to construct the recombinant cloning vector DBN100014 containing Cry1Fa-01 nucleotide sequence for pest control in this invention;

FIG. 3 shows the control effect of transgenic corn plants against Sesamia inferen pests in this invention;

FIG. 4 shows the control effect of transgenic rice plants against Sesamia inferen pests in this invention.

### DETAILED DESCRIPTION OF THE INVENTION

The technical solutions of this invention for controlling pests will be further illustrated through the following examples.

### Example 1

### The Obtaining and Synthesis of Cry1Fa Gene

### 1. Obtaining of Cry1Fa Nucleotide Sequence

Amino acid sequence of Cry1Fa-01 pesticidal protein (605 amino acids) was shown as SEQ ID NO: 1 in the sequence listing; Nucleotide sequence of Cry1Fa-01 gene (1818 nucleotides) encoding the corresponding amino acid sequence of Cry1Fa-01 pesticidal protein (605 amino acids) was shown as SEQ ID NO: 3 in the sequence listing; Amino acid sequence of Cry1Fa-02 pesticidal protein (1148 amino acids) was shown as SEQ ID NO: 2 in the sequence listing; the nucleotide sequence of Cry1Fa-02 gene (3447 nucleotides) encoding the corresponding amino acid sequence of Cry1Fa-02 pesticidal protein (1148 amino acids) was shown as SEQ ID NO: 4 in the sequence listing.

### 2. Obtaining of Cry1Ab and Vip3A Nucleotide Sequences

Nucleotide sequence of Cry1Ab (1848 nucleotides) encoding the corresponding amino acid sequence of Cry1Ab pesticidal protein (615 amino acids) was shown as SEQ ID NO: 5 in the sequence listing and nucleotide sequence of Vip3A (2370 nucleotides) encoding the corresponding amino acid sequence of Vip3A pesticidal protein (789 amino acids) was shown as SEQ ID NO: 6 in the sequence listing.

### 3. Synthesis of the Nucleotide Sequence as Described Above

The Cry1Fa-01 nucleotide sequence (shown as SEQ ID NO: 3 in the sequence listing), Cry1Fa-02 nucleotide sequence (shown as SEQ ID NO: 4 in the sequence listing), Cry1Ab nucleotide sequence (shown as SEQ ID NO: 5 in the sequence listing) and Vip3A nucleotide sequence (shown as SEQ ID NO: 6 in the sequence listing) were synthesized by GenScript CO., LTD, Nanjing, P.R. China. The synthesized Cry1Fa-01 nucleotide sequence (SEQ ID NO: 3) was linked with an AscI restriction site at the 5′ end and a BamHI restriction site at the 3′ end. The synthesized Cry1Fa-02 nucleotide sequence (SEQ ID NO: 4) was linked with an AscI restriction site at the 5′ end and a BamHI restriction site at the 3′ end. The synthesized Cry1Ab nucleotide sequence (SEQ ID NO: 5) was linked with a NcoI restriction site at the 5′ end and a SwaI restriction site at the 3′ end. The synthesized Vip3A nucleotide sequence (SEQ ID NO: 6) was linked with a ScaI restriction site at the 5′ end and a SpeI restriction site at the 3′ end.

### Example 2

### Construction of Recombinant Expression Vectors and the Transfection of Agrobacterium with the Recombinant Expression Vectors

### 1. Construction of the Recombinant Cloning Vectors Containing Cry1F Gene

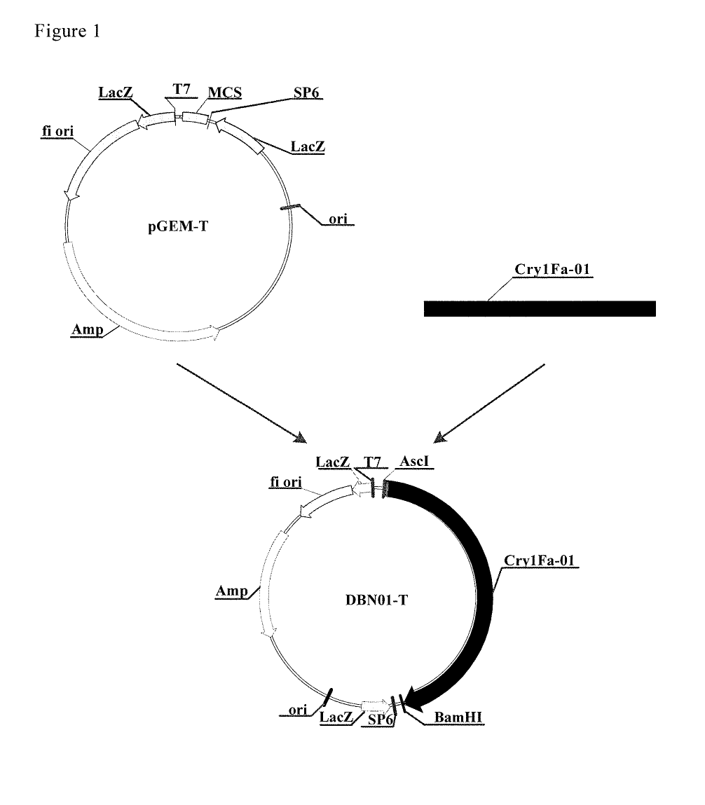

The synthesized Cry1Fa-01 nucleotide sequence was sub-cloned into cloning vector pGEM-T (Promega, Madison, USA, CAT: A3600), to get cloning vector DBN01-T following the instructions of Promega pGEM-T vector, and the construction process was shown in FIG. 1 (wherein the Amp is ampicillin resistance gene; f1 is the replication origin of phage f1; LacZ is initiation codon of LacZ; SP6 is the promoter of SP6 RNA polymerase; T7 is the promoter of T7 RNA polymerase; Cry1Fa-01 is Cry1Fa-01 nucleotide sequence (SEQ ID NO: 3); MCS is multiple cloning sites).

The recombinant cloning vector DBN01-T was then transformed into E. coli T1 competent cell (Transgen, Beijing, China, the CAT: CD501) through heat shock method. The heat shock conditions were as follows: 50 μl of E. coli T1 competent cell and 10 μl of plasmid DNA (recombinant cloning vector DBN01-T) were incubated in water bath at 42° C. for 30 seconds. Then the E. coli cells were incubated in water bath at 37° C. for 1 h (100 rpm in a shaking incubator) and then were grown on a LB plate (10 g/L Tryptone, 5 g/L yeast extract, 10 g/L NaCl, 15 g/L Agar and pH was adjusted to 7.5 with NaOH) coated on the surface with IPTG (Isopropyl thio-beta-D-galactoseglucoside), X-gal (5-bromine-4-chlorine-3-indole-beta-D-galactose glucoside) and ampicillin (100 mg/L) overnight. The white colonies were picked out and cultivated in LB broth (10 g/L Tryptone, 5 g/L yeast extract, 10 g/L NaCl, 100 mg/L ampicillin and pH was adjusted to 7.5 with NaOH) at 37° C. overnight. The plasmids thereof were extracted using alkaline lysis method as follows: the cell broth was centrifuged for 1 mM at 12000 rpm, the supernatant was discarded and the pellet was resuspended in 100 μl of ice-chilled solution I (25 mM Tris-HCl, 10 mM EDTA (ethylenediaminetetraacetic acid) and 50 mM glucose, pH 8.0); then 150 μl of freshly prepared solution II (0.2 M NaOH, 1% SDS (sodium dodecyl sulfate)) was added and the tube was reversed 4 times, mixed and then put on ice for 3-5 mM; 150 μl of cold solution III (4 M potassium acetate and 2 M acetic acid) was added, thoroughly mixed immediately and incubated on ice for 5-10 mM; the mixture was centrifuged at 12000 rpm at 4° C. for 5 min, two volumes of anhydrous ethanol were added into the supernatant, mixed and then placed at room temperature for 5 mM; the mixture was centrifuged at 12000 rpm at 4° C. for 5 min, the supernatant was discarded and the pellet was dried after washed with 70% ethanol (V/V); 30 μl TE (10 mM Tris-HCl, 1 mM EDTA, pH 8.0) containing RNase (20 μg/ml) was added to dissolve the precipitate; the mixture was incubated at 37° C. in a water bath for 30 min to digest RNA and stored at −20° C., for the future use.

After the extracted plasmids were confirmed with restriction enzymes AscI and BamHI, the positive clones were verified through sequencing. The results showed that the Cry1Fa-01 nucleotide sequence inserted into the recombinant cloning vector DBN01-T was the sequence set forth in SEQ ID NO: 3 in the sequence listing, indicating that Cry1Fa-01 nucleotide sequence was correctly inserted.

The synthesized nucleotide sequence Cry1Fa-02 was inserted into cloning vector pGEM-T to get recombinant cloning vector DBNO2-T following the process for constructing cloning vector DBN01-T as described above, wherein Cry1Fa-02 was Cry1Fa-02 nucleotide sequence (SEQ ID NO: 4). The Cry1Fa-02 nucleotide sequence in the recombinant cloning vector DBNO2-T was verified to be correctly inserted with restriction enzyme digestion and sequencing.

The synthesized nucleotide sequence Cry1Ab was inserted into cloning vector pGEM-T to get recombinant cloning vector DBNO3-T following the process for constructing cloning vector DBN01-T as described above, wherein Cry1Ab was Cry1Ab nucleotide sequence (SEQ ID NO: 5). The Cry1Ab nucleotide sequence in the recombinant cloning vector DBNO3-T was verified to be correctly inserted with restriction enzyme digestion and sequencing.

The synthesized nucleotide sequence Vip3A was inserted into cloning vector pGEM-T to get recombinant cloning vector DBN04-T following the process for constructing cloning vector DBN01-T as described above, wherein Vip3A was Vip3A nucleotide sequence (SEQ ID NO: 6). The Vip3A nucleotide sequence in the recombinant cloning vector DBN04-T was verified to be correctly inserted with restriction enzyme digestion and sequencing.

### 2. Construction of the Recombinant Expression Vectors Containing Cry1F Gene

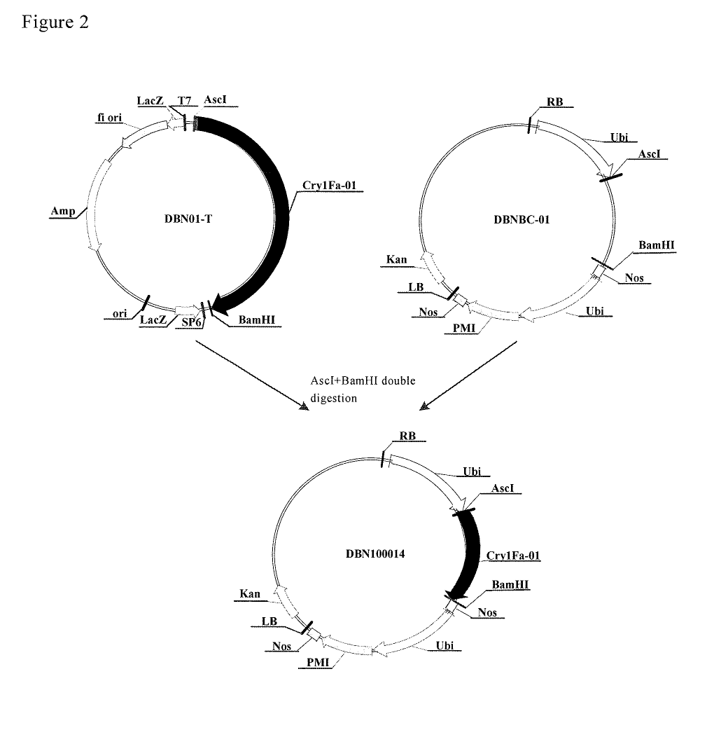

The recombinant cloning vector DBN01-T and expression vector DBNBC-01 (Vector backbone: pCAMBIA2301, available from CAMBIA institution) were digested with restriction enzymes AscI and BamHI. The cleaved Cry1Fa-01 nucleotide sequence fragment was ligated between the restriction sites AscI and BamHI of the expression vector DBNBC-01 to construct the recombinant expression vector DBN100014. It is a well-known conventional method to construct expression vector through restriction enzyme digestion. The construction scheme was shown in FIG. 2 (Kan: kanamycin gene; RB: right border; Ubi: maize Ubiquitin (Ubiquitin) gene promoter (SEQ ID NO: 7); Cry1Fa-01: Cry1Fa-01 nucleotide sequence (SEQ ID NO: 3); Nos, terminator of nopaline synthetase gene (SEQ ID NO: 8); PMI: phosphomannose isomerase gene (SEQ ID NO: 9); LB: left border).

The recombinant expression vector DBN100014 was transformed into E. coli T1 competent cells with heat shock method as follows: 50 μl of E. coli T1 competent cell and 10 μl of plasmid DNA (recombinant expression vector DBN100014) were incubated in water bath at 42° C. for 30 seconds. Then the E. coli cells were incubated in water bath at 37° C. for 1 h (100 rpm in a shaking incubator) and then were grown on a LB solid plate (10 g/L Tryptone, 5 g/L yeast extract, 10 g/L NaCl, 15 g/L Agar and pH was adjusted to 7.5 with NaOH) containing 50 mg/L kanamycin at 37° C. for 12 hrs. The white colonies were picked out and cultivated in LB broth (10 g/L Tryptone, 5 g/L yeast extract, 10 g/L NaCl, 50 mg/L kanamycin and pH was adjusted to 7.5 with NaOH) at 37° C. overnight. The plasmids thereof were extracted using alkaline lysis method. After the extracted plasmids were confirmed with restriction enzymes AscI and BamHI, the positive clones were verified through sequencing. The results showed that the nucleotide sequence between restriction sites AscI and BamHI in the recombinant expression vector DBN100014 was the nucleotide sequence set forth in SEQ ID NO: 3 in the sequence listing, i.e. Cry1Fa-01 nucleotide sequence.

Following the process for constructing recombinant expression vector DBN100014 as described above, recombinant cloning vectors DBN01-T and DBNO3-T were digested with restriction enzymes AscI/BamHI and NcoI/SwaI respectively to cleave the Cry1Fa-01 nucleotide sequence and Cry1Ab nucleotide sequence which then were inserted into the expression vector DBNBC-01 to get the recombinant expression vector DBN100012. Restriction enzyme digestion and sequencing verified that recombinant expression vector DBN100012 contained the nucleotide sequences set forth in SEQ ID NO: 3 and SEQ ID NO: 5 in the sequence listing, i.e. the nucleotide sequences of Cry1Fa-01 and Cry1Ab.

Following the process for constructing recombinant expression vector DBN100014 as described above, recombinant cloning vectors DBNO2-T and DBN04-T were digested with restriction enzymes AscI/BamHI and ScaI/SpeI respectively to cleave the Cry1Fa-02 nucleotide sequence and Vip3A nucleotide sequence which then were inserted into the expression vector DBNBC-01 to get the recombinant expression vector DBN100276. Restriction enzyme digestion and sequencing verified that recombinant expression vector DBN100276 contained the nucleotide sequences set forth in SEQ ID NO: 4 and SEQ ID NO: 6 in the sequence listing, i.e. the nucleotide sequences of Cry1Fa-02 and Vip3A.

3. Transfection of Agrobacterium turnefaciens with the Recombinant Expression Vectors

The correctly constructed recombinant expression vectors DBN100014, DBN100012 and DBN100276 were transfected into Agrobacterium LBA4404 (Invitrgen, Chicago, USA, Cat. No: 18313-015) following liquid nitrogen rapid-freezing method as follows: 100 μL Agrobacterium LBA4404 and 3 μL plasmid DNA (recombinant expression vector) were put into liquid nitrogen for 10 min and then incubated in water bath at 37° C. for 10 min. Then the transfected Agrobacterium LBA4404 cells were inoculated in LB broth and cultivated at 28° C., 200 rpm for 2 hours and spraid on a LB plate containing 50 mg/L of rifampicin (Rifampicin) and 100 mg/L of kanamycin (Kanamycin) until positive mono colonies appeared. The positive mono colonies were picked up and cultivated and the plasmids thereof were extracted. Recombinant expression vectors DBN100014 and DBN100012 were verified with restriction enzymes AhdI and XbaI and recombinant expression vector DBN100276 was verified with restriction enzymes AhdI and AatII. The results showed that the recombinant expression vectors DBN100014, DBN100012 and DBN100276 were correct in structure, respectively.

### Example 3

### Obtaining and Verification of the Transgenic Corn Plant with Inserted Cry1F Gene

1. Obtaining of the Transgenic Corn Plant with Inserted Cry1F Gene

According to the conventional Agrobacterium transfection method, the maize cultivar Zong 31 (Z31) was cultivated in sterilized conditions and the young embryo was co-cultivated with the Agrobacterium strains constructed in part 3 of Example 2 so as to introduce T-DNAs in the recombinant expression vectors DBN100014, DBN100012 and DBN100276 constructed in part 2 of Example 2 (including corn Ubiquitin gene promoter sequence, Cry1Fa-01 nucleotide sequence, Cry1Fa-02 nucleotide sequence, Cry1Ab nucleotide sequence, Vip3A nucleotide sequence, PMI gene and Nos terminator sequence) into the maize genome. Maize plants containing Cry1Fa-01 nucleotide sequence, maize plants containing Cry1Fa-01-Cry1Ab nucleotide sequence and maize plants containing Cry1Fa-02-Vip3A nucleotide sequence were obtained respectively and wild type corn plant was taken as a control.

As to the Agrobacterium-mediated transfection of maize, in brief, immature maize young embryo was isolated from corns and contacted with Agrobacterium suspension, in which the Agrobacterium can deliver the Cry1Fa-01 nucleotide sequence, Cry1Fa-01-Cry1Ab nucleotide sequence or Cry1Fa-02-Vip3A nucleotide sequence into at least one cell of one young embryo. (Step 1: infection step). In this step, preferably, young embryo was immersed in Agrobacterium suspension (OD₆₆₀=0.4˜0.6, infection medium (4.3 g/L of MS salt, MS vitamins, 300 mg/L of casein, 68.5 g/L of sucrose, 36 g/L of glucose, 40 mg/L of Acetosyringone (AS), 1 mg/L of 2,4-dichlorophenoxyacetic acid (2,4-D), pH=5.3)) to initiate the inoculation. Young embryo and Agrobacterium were cocultivated for a period (3 days) (Step 2: cocultivation step). Preferably, the Young embryo was cultivated on a solid medium (4.3 g/L of MS salt, MS vitamins, 300 mg/L of casein, 20 g/L of sucrose, 10 g/L of glucose, 100 mg/L of Acetosyringone (AS), 1 mg/L of 2,4-dichlorophenoxyacetic acid (2,4-D) and 8 g/L of Agar, pH=5.8) after the infection step. After this cocultivation step, a selective “recovery” step can be preceded. In the “recovery” step, the recovery medium (4.3 g/L of MS salt, MS vitamins, 300 mg/L of casein, 30 g/L of sucrose, 1 mg/L of 2,4-dichlorophenoxyacetic acid (2,4-D) and 8 g/L of Agar, pH=5.8) contains at least one kind of known Agrobacterium-inhibiting antibiotics (cephamycin) without the selective agent for plant transfectants (Step 3: recovery step). Preferably, the young embryo was cultivated on a solid medium culture containing antibiotics but without selective agent so as to eliminate Agrobacterium and to provide a recovery period for the infected cells. Then, the inoculated young embryo was cultivated on a medium containing selective agent (mannose) and the transfected callus was selected (Step 4: selection step). Preferably, the young embryo was cultivated on a selective solid medium containing selective agent (4.3 g/L of MS salt, MS vitamins, 300 mg/L of casein, 5 g/L of sucrose, 12.5 g/L of mannose, 1 mg/L of 2,4-dichlorophenoxyacetic acid (2,4-D) and 8 g/L of Agar, pH=5.8), resulting the selective growth of the transfected cells. Then, callus regenerated into plants (Step 5: regeneration step). Preferably, the callus was cultivated on a solid medium containing selective agent (MS differentiation medium and MS rooting medium) to regenerate into plants.

The obtained resistant callus was transferred to the MS differentiation medium (4.3 g/L MS salt, MS vitamins, 300 mg/L of casein, 30 g/L of sucrose, 2 mg/L of 6-benzyladenine, 5 g/L of mannose and 8 g/L of Agar, pH=5.8) and cultivated and differentiated at 25° C. The differentiated seedlings were transferred to the MS rooting medium (2.15 g/Lof MS salt, MS vitamins, 300 mg/L of casein 30 g/L of sucrose, 1 mg/L indole-3-acetic acid and 8 g/L of agar, pH=5.8) and cultivated to about 10 cm in height at 25 T. Next, the seedlings were transferred to and cultivated in the greenhouse until fructification. In the greenhouse, the maize plants were cultivated at 28° C. for 16 hours and at 20° C. for 8 hours every day.

2. Verification of Transgenic Corn Plants with Inserted Cry1F Gene Using TaqMan Technique

100 mg of leaves from every transfected corn plant (corn plant transfected with Cry1Fa-01 nucleotide sequence, Cry1Fa-01-Cry1Ab nucleotide sequence or Cry1Fa-02-Vip3A nucleotide sequence, respectively) was taken as sample respectively. Genomic DNA thereof was extracted using DNeasy Plant Maxi Kit (Qiagen) and the copy numbers of Cry1F gene, Cry1Ab gene and Vip3A gene were quantified through Taqman probe-based fluorescence quantitative PCR assay. Wild type maize plant was taken as a control and analyzed according to the processes as described above. Experiments were carried out in triplicate and the results were the mean values.

The specific method for detecting the copy numbers of Cry1F gene, Cry1Ab gene and Vip3A gene was described as follows.

Step 11: 100 mg of leaves from every transfected corn plant (corn plant transfected with nucleotide sequence of Cry1Fa-01, Cry1Fa-01-Cry1Ab or Cry1Fa-02-Vip3A, respectively) was taken and grinded into homogenate in a mortar in liquid nitrogen respectively. It was in triplicate for each sample.

Step 12: the genomic DNAs of the samples above were extracted using DNeasy Plant Mini Kit (Qiagen) following the product instruction thereof.

Step 13: the genome DNA concentrations of the above samples were determined using NanoDrop 2000 (Thermo Scientific).

Step 14: the genome DNA concentrations were adjusted to the same range of 80-100 ng/μl.

Step 15: the copy numbers of the samples were quantified using Taqman probe-based fluorescence quantitative PCR assay, the quantified sample with known copy number was taken as a standard sample and the wild type maize plant was taken as a control. It was carried out in triplicate for every sample and the results were the mean values. Primers and the probes used in the fluorescence quantitative PCR were shown as below.

The following primers and probe were used to detect Cry1Fa-01 nucleotide sequence:

Primer 1 (CF1): CAGTCAGGAACTACAGTTGTAAGAGGG (as shown in SEQ ID NO: 10 in the sequence listing);
Primer 2 (CR1): ACGCGAATGGTCCTCCACTAG (as shown in SEQ ID NO: 11 in the sequence listing);
Probe 1 (CP1): CGTCGAAGAATGTCTCCTCCCGTGAAC (as shown in SEQ ID NO: 12 in the sequence listing)

The following primers and probe were used to detect Cry1Ab nucleotide sequence:

Primer 3 (CF2): TGGTGGAGAACGCATTGAAAC (as shown in SEQ ID NO: 13 in the sequence listing);
Primer 4 (CR2): GCTGAGCAGAAACTGTGTCAAGG (as shown in SEQ ID NO: 14 in the sequence listing);
Probe 2 (CP2): CGGTTACACTCCCATCGACATCTCCTTG (as shown in SEQ ID NO: IS in the sequence listing);

The following primers and probe were used to detect Cry1Fa-02 nucleotide sequence:

Primer 5 (CF3): CAGTCAGGAACTACAGTTGTAAGAGGG (as shown in SEQ ID NO: 16 in the sequence listing);
Primer 6 (CR3): ACGCGAATGGTCCTCCACTAG (as shown in SEQ ID NO: 17 in the sequence listing);
Probe 3 (CP3): CGTCGAAGAATGTCTCCTCCCGTGAAC (as shown in SEQ ID NO: 18 in the sequence listing)

The following primers and probe were used to detect Vip3A nucleotide sequence:

Primers 7 (CF4): ATTCTCGAAATCTCCCCTAGCG (as shown in SEQ ID NO: 19 in the sequence listing);
Primer 8 (CR4): GCTGCCAGTGGATGTCCAG (as shown in SEQ ID NO: 20 in the sequence listing);
Probe 4 (CP4): CTCCTGAGCCCCGAGCTGATTAACACC (as shown in SEQ ID NO: 21 in the sequence listing)

PCR reaction system was as follows:

| | |
|---|---|
| JumpStart ™ Taq ReadyMix ™ (Sigma) | 10 μl |
| 50X primer/probe mixture | 1 μl |
| Genomic DNA | 3 μl |
| Water (ddH₂O) | 6 μl |

The 50× primer/probe mixture contained 45 μl of each primer (1 mM), 50 μl of probe (100 μM) and 860 μl of 1×TE buffer and was stored in an amber tube at 4° C.

PCR reaction conditions were provided as follows:

| Step | Temperature | Time |
|---|---|---|
| 21 | 95° C. | 5 min |
| 22 | 95° C. | 30 s |
| 23 | 60° C. | 1 min |

| | |
|---|---|
| 24 | back to step 22 and repeated 40 times |

Data were analyzed using software SDS 2.3 (Applied Biosystems).

The experimental results showed that all the nucleotide sequences of Cry1Fa-01, Cry1Fa-01-Cry1Ab and Cry1Fa-02-Vip3A have been integrated into the genomes of the detected corn plants, respectively. Furthermore, corn plants transfected with nucleotide sequences of Cry1Fa-01, Cry1Fa-01-Cry1Ab and Cry1Fa-02-Vip3A respectively contained single copy of Cry1F gene, Cry1Ab gene, and/or Vip3A gene respectively.

### Example 4

### Detection of Pesticidal Protein in Transgenic Corn Plants

### 1. Content Detection of the Pesticidal Protein in Transgenic Corn Plants

Solutions involved in this experiment were as follows:

Extraction buffer: 8 g/L of NaCl, 0.2 g/L of KH₂PO₄, 2.9 g/L of Na₂HPO₄.12H₂O, 0.2 g/L of KCl, 5.5 ml/L of Tween-20, pH=7.4;
Washing buffer PBST: 8 g/L of NaCl, 0.2 g/L of KH₂PO₄, 2.9 g/L of Na₂HPO₄.12H₂O, 0.2 g/L of KCl, 0.5 ml/L of Tween-20, pH=7.4;
Stop solution: 1 M HCl.

3 mg of fresh leaves from every transfected corn plant (corn plant transfected with nucleotide sequence of Cry1Fa-01, Cry1Fa-01-Cry1Ab or Cry1Fa-02-Vip3A, respectively) was taken as a sample respectively. All the samples were grinded in liquid nitrogen and 800 μl of the extraction solution was added therein. The mixture was centrifuged at 4000 rpm for 10 min and the supernatant was diluted 40 folds with the extraction buffer and 80 μl of the diluted supernatant was taken out for an ELISA test. The ratio of pesticidal protein (Cry1Fa protein, Cry1Ab protein and Vip3A protein)/fresh weight of leaves was determined using an ELISA (enzyme-linked immunosorbent assay) kit (ENVIRLOGIX Co., Cry1Fa kit, Cry1Ab kit and Vip3A kit) and the specific method was shown in the product instruction.

At the same time, the wild type maize plants and the maize plants identified as non-transgenic maize plants with the Taqman technique were taken as controls and analyzed following the above methods. There were three strains (S1, S2, and S3) containing the inserted nucleotide sequence Cry1Fa-01, three strains (S4, S5 and S6) containing the inserted nucleotide sequence Cry1F-01-Cry1Ab and three strains (S7, S8 and S9) containing the inserted nucleotide sequence Cry1Fa-02-Vip3A. There presented one strain identified as non-transgenic (NGM1) via Taqman technique and one wild type strain (CK1). Three plants of each strain were selected for further tests and each plant was repeated 6 times.

Pesticidal protein (Cry1Fa protein) contents in the transgenic maize plants were shown in Table 1. Pesticidal protein (Cry1Ab protein) contents in the transgenic maize plants were shown in Table 2. Pesticidal protein (Vip3A protein) contents in the transgenic maize plants were shown in Table 3. Ratios (ng/g) of the average expression value of the pesticidal protein (Cry1Fa protein) vs fresh weight of the leaves of the corn plants containing nucleotide sequence of Cry1Fa-01, Cry1Fa-01-Cry1Ab or Cry1Fa-02-Vip3A were 3475.52, 3712.48 or 3888.76 respectively. Ratio (ng/g) of the average expression value of the pesticidal protein (Cry1Ab protein) vs fresh weight of the leaves of the corn plant containing nucleotide sequence Cry1Fa-01-Cry1Ab was 8234.7. Ratio (ng/g) of the average expression value of the pesticidal protein (Vip3A protein) vs fresh weight of the leaves of the corn plant containing nucleotide sequence Cry1Fa-02-Vip3A was 3141.02. These results showed that all Cry1Fa protein, Cry1Ab protein and Vip3A protein were expressed highly and stably in maize plants.

TABLE 1
Average expression values of Cry1Fa protein in transgenic corn plants

| | Expression values | | | |
|---|---|---|---|---|
| | of Cry1Fa protein in | | | |
| | a single plant (ng/g) | | | |
| | (repeated 6 times | | | Expression values of Cry1Fa |
| | for each plant) | | | protein in each strain (ng/g) |
| Strain | 1 | 2 | 3 | Average expression value (ng/g) |
| S1 | 3535.02 | 3697.34 | 2928.71 | 3475.52 |
| S2 | 3904.88 | 2808.72 | 3044.88 | |
| S3 | 3954.63 | 3572.96 | 3832.55 | |
| S4 | 3039.78 | 3600.01 | 3753.22 | 3712.48 |
| S5 | 4543.98 | 4251.25 | 3862.03 | |
| S6 | 3049.4 | 3834.01 | 3478.66 | |
| S7 | 3892.15 | 4215.07 | 3941.55 | 3888.76 |
| S8 | 3905.47 | 3816.27 | 4028.96 | |
| S9 | 3617.49 | 3795.65 | 3786.19 | |
| NGM1 | −0.23 | 0 | −4.21 | 0 |
| CK1 | −2.36 | −1.98 | 0 | 0 |

TABLE 2
Average expression values of Cry1Ab protein in transgenic corn plants

| | Expression values | | | |
|---|---|---|---|---|
| | of Cry1Ab protein | | | |
| | in a single plant | | | |
| | (ng/g) (repeated | | | |
| | 6 times | | | Expression values of Cry1Ab |
| | for each plant) | | | protein in each strain (ng/g) |
| Strain | 1 | 2 | 3 | Average expression value (ng/g) |
| S4 | 7088.4 | 9837.5 | 10626.4 | 8234.7 |
| S5 | 9866.7 | 6863.3 | 4222.4 | |
| S6 | 9912.1 | 7724.1 | 7970.9 | |
| NGM1 | −4.51 | −2.44 | 0 | 0 |
| CK1 | 0 | −6.33 | −1.97 | 0 |

TABLE 3
Average expression values of Vip3A protein in transgenic corn plants

| | Expression values | | | |
|---|---|---|---|---|
| | of Vip3A protein in a | | | |
| | single plant (ng/g) | | | Expression values of Vip3A |
| | (repeated 6 times | | | protein in each strain (ng/g) |
| | for each plant) | | | Average expression values |
| Strain | 1 | 2 | 3 | (ng/g) |
| S7 | 2989.67 | 3123.65 | 3176.48 | 3141.02 |
| S8 | 3205.68 | 3102.69 | 3312.03 | |
| S9 | 3059.11 | 3246.85 | 3167.95 | |
| NGM1 | −1.52 | 0 | −6.34 | 0 |
| CK1 | 0 | −0.95 | −2.31 | 0 |

### 1. Insect-Resistance Effects Test of the Transgenic Corn Plants

Sesamia inferen-resistance effect of the corn plants transfected with Cry1Fa-01 nucleotide sequence, corn plants transfected with Cry1Fa-01-Cry1Ab nucleotide sequence, corn plants transfected with Cry1Fa-02-Vip3A nucleotide sequence, the wild type corn plants and corn plants identified as non-transgenic with Taqman technique were tested.

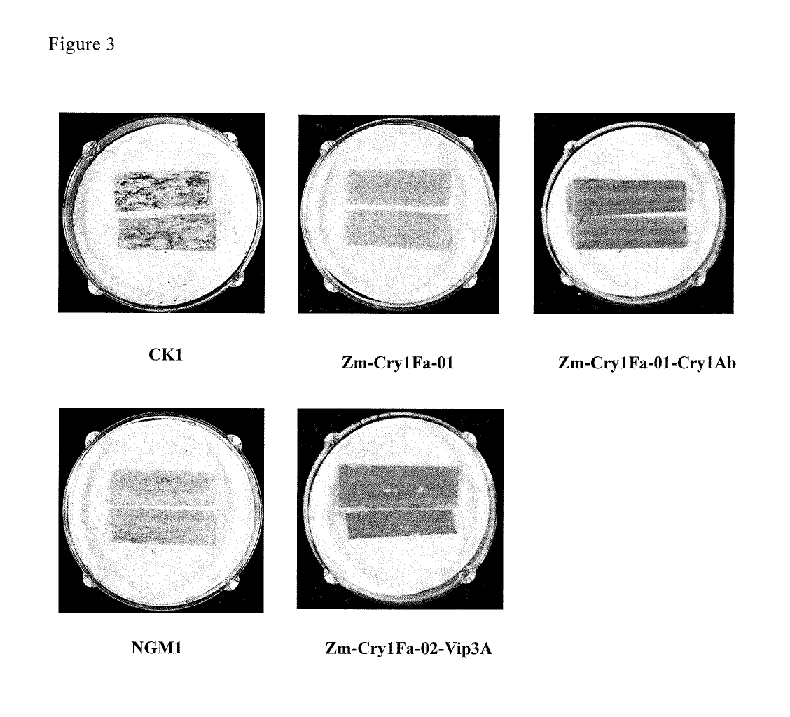

Fresh leaves of the corn plants transfected with Cry1Fa-01 nucleotide sequence, Cry1Fa-01-Cry1Ab nucleotide sequence or Cry1Fa-02-Vip3A nucleotide sequence, the wild type corn plants and corn plants identified as non-transgenic with Taqman technique (stages V6-V8) were taken respectively and washed with sterile water, and the water remained on the leaf surfaces were dried with a piece of gauze. The leaf veins were removed and at the same time the leaves were cut into long strips (1 cm*2 cm). Two strips were put on a filter paper on the bottom of a round plastic Petri dish. The filter paper was wet with distilled water and 10 artificially fed Sesamia inferens (newly hatched larvae) were put in each round plastic Petri dish. Then the Petri dish was covered and kept for 3 days in a condition with a temperature of 26-28° C., relative humidity 70%-80%, photoperiod (light/dark)16:8. Then, statistics of leaf feeding, larvae survival and development conditions were carried out, and average corrected mortality and larvae weight from every sample were calculated. Average corrected mortality M=(Mt−Mc)/(1−Mc)*100%, wherein M is average corrected mortality (%), Mt is the average mortality (%) of the insects on corn plants to be tested, Mc is the average mortality (%) of the insects on the control plants (CK1). The insect-resistance grading standard was shown in Table 4. Three strains (S1, S2, and S3) of corn plants transfected with Cry1Fa-01 nucleotide sequence; three strains (S4, S5, and S6) of corn plants transfected with Cry1F-01-Cry1Ab nucleotide sequence; three strains (S7, S8, and S9) of corn plants transfected with Cry1Fa-02-Vip3A nucleotide sequence; one strain identified as non-transgenic (NGM1) via Taqman technique and one wild type strain (CK1) were selected. Three plants of each strain were tested and each plant is repeated 6 times. The results were shown in Table 5 and FIG. 3.

TABLE 4
Insect-resistance grading standard

| | Corrected mortality (%), |
|---|---|
| Grading | development condition |
| HR (highly resistant) | 85.1-100, Survived test insects scarcely |
| | developed |
| R (resistant) | 60.1-85, or development of the survived test |
| | insects were obviously delayed |
| MR (moderately resistant) | 40.1-60, or survived test insects |
| | developed while their development was |
| | somewhat delayed. |
| MS(moderately susceptible) | 20.1-40, and development of the |
| | survived test insects was substantially |
| | normal. |
| S (susceptible) | <20, and development of the survived test |
| | insects was normal |

TABLE 5
Insect-resistances of the transgenic corn plants inoculated
with Sesamia inferens

| | | | Total | | | | |
|---|---|---|---|---|---|---|---|
| | | | weight of | | | | |
| | Larvae numbers | | the | | | | |
| | Inoc- | Sur- | survived | Corrected | | Weight/each | |
| | ulated | vived | larvae | mortality | | insect | |
| | larvae | larvae | (mg) | (%) | Average | (mg) | Average |
| S1-1 | 10 | 0 | 0 | 100 | 94.1 | 0 | 0.15 |
| S1-2 | 10 | 0 | 0 | 100 | | 0 | |
| S1-3 | 10 | 0 | 0 | 100 | | 0 | |
| S2-1 | 10 | 1 | 0.2 | 89.3 | | 0.20 | |
| S2-2 | 10 | 1 | 0.1 | 89.3 | | 0.10 | |
| S2-3 | 10 | 2 | 0.4 | 78.5 | | 0.20 | |
| S3-1 | 10 | 0 | 0 | 100 | | 0 | |
| S3-2 | 10 | 0 | 0 | 100 | | 0 | |
| S3-3 | 10 | 1 | 0.1 | 89.3 | | 0.10 | |
| S4-1 | 10 | 2 | 0.1 | 78.5 | 91.7 | 0.10 | 0.15 |
| S4-2 | 10 | 1 | 0.2 | 89.3 | | 0.20 | |
| S4-3 | 10 | 0 | 0 | 100 | | 0 | |
| S5-1 | 10 | 0 | 0 | 100 | | 0 | |
| S5-2 | 10 | 2 | 0.2 | 78.5 | | 0.10 | |
| S5-3 | 10 | 0 | 0 | 100 | | 0 | |
| S6-1 | 10 | 0 | 0 | 100 | | 0 | |
| S6-2 | 10 | 2 | 0.3 | 78.5 | | 0.15 | |
| S6-3 | 10 | 0 | 0 | 100 | | 0 | |
| S7-1 | 10 | 0 | 0 | 100.0 | 92.9 | 0 | 0.12 |
| S7-2 | 10 | 1 | 0.1 | 89.3 | | 0.10 | |
| S7-3 | 10 | 1 | 0.2 | 89.3 | | 0.20 | |
| S8-1 | 10 | 0 | 0 | 100.0 | | 0 | |
| S8-2 | 10 | 1 | 0.1 | 89.3 | | 0.10 | |
| S8-3 | 10 | 0 | 0 | 100.0 | | 0 | |
| S9-1 | 10 | 2 | 0.2 | 78.5 | | 0.10 | |
| S9-2 | 10 | 1 | 0.1 | 89.3 | | 0.10 | |
| S9-3 | 10 | 0 | 0 | 100.0 | | 0 | |
| NGM1- | 10 | 8 | 102.2 | 14.0 | 10.4 | 12.78 | 15.32 |
| 1 | | | | | | | |
| NGM1- | 10 | 8 | 128.9 | 14.0 | | 16.11 | |
| 2 | | | | | | | |
| NGM1- | 10 | 9 | 153.6 | 3.2 | | 17.07 | |
| 3 | | | | | | | |
| CK1-1 | 10 | 9 | 114.4 | | 0 | 12.71 | 16.31 |
| CK1-2 | 10 | 10 | 189.3 | | | 18.93 | |
| CK1-3 | 10 | 9 | 155.5 | | | 17.28 | |

Results of Table 5 and FIG. 3 showed that average corrected mortalities of most corn plants transfected with the Cry1Fa-01 nucleotide sequence, corn plants transfected with the Cry1Fa-01-Cry1Ab and corn plants transfected with the Cry1Fa-02-Vip3A were around or above 90%, and average corrected mortalities of some strains were up to 100%. Compared with this, the average corrected mortalities of wild type corn plants were generally round or below 10%. Compared with the wild type corn plants, control efficiencies against newly hatched larvae of corn plants transfected with the Cry1Fa-01 nucleotide sequence, corn plants transfected with the Cry1Fa-01-Cry1Ab and corn plants transfected with the Cry1Fa-02-Vip3A were almost 100% and the individual larvae scarcely survived also substantially stopped development. Furthermore, corn plants transfected with the Cry1Fa-01 nucleotide sequence, corn plants transfected with the Cry1Fa-01-Cry1Ab and corn plants transfected with the Cry1Fa-02-Vip3A were only slightly harmed in general.

It was thereby demonstrated that all corn plants transfected with the Cry1Fa-01 nucleotide sequence, corn plants transfected with the Cry1Fa-01-Cry1Ab and corn plants transfected with the Cry1Fa-02-Vip3A showed high Sesamia inferen-resistant activity, which was enough to result in a harmful effect to the growth of Sesamia inferen and to control Sesamia inferen.

### Example 5

### Obtaining and Verification of the Transgenic Rice Plant with Inserted Cry1F Gene

1. Obtaining of the Transgenic Rice Plant with Inserted Cry1F Gene

According to the conventional Agrobacterium transfection method, the japonica rice Nipponbare was cultivated in sterilized conditions and the young embryo was co-cultivated with the Agrobacterium strains constructed in part 3 of Example 2 so as to introduce T-DNAs in the recombinant expression vectors DBN100014, DBN100012 and DBN100276 constructed in part 2 of Example 2 (including corn Ubiquitin gene promoter sequence, nucleotide sequences of Cry1Fa-01 nucleotide sequence, Cry1Fa-02 nucleotide sequence, Cry1Ab nucleotide sequence, Vip3A nucleotide sequence, PMI gene and Nos terminator sequence) into the rice genome. Rice plants containing Cry1Fa-01 nucleotide sequence, rice plants containing Cry1Fa-01-Cry1Ab nucleotide sequence and rice plants containing Cry1Fa-02-Vip3A nucleotides sequence were obtained respectively and wild type rice plant was taken as a control.

Regarding to the Agrobacterium-mediated transfection of rice, briefly, rice seeds were inoculated on induction medium (N6 salt, N6 vitamins, 300 mg/L of casein, 30 g/L of sucrose, 2 mg/L of 2,4-dichlorophenoxyacetic acid (2,4-D) and 3 g/L of plant gelatum, pH=5.8) and callus was induced from mature embryo of rice (Step 1: callus induction step). Then the next is to optimize callus. Callus was contacted with Agrobacterium suspension, in which the Agrobacterium can deliver the Cry1Fa-01 nucleotide sequence, Cry1Fa-01-Cry1Ab nucleotide sequence or Cry1Fa-02-Vip3A nucleotide sequence into at least one cell of the callus (Step 2: infection step). In this step, preferably, callus was immersed in Agrobacterium suspension (OD₆₆₀=0.3, infection medium (N6 salt, N6 vitamins, 300 mg/L of casein, 30 g/L of sucrose, 10 g/L of glucose, 40 mg/L of Acetosyringone (AS), 2 mg/L of 2,4-dichlorophenoxyacetic acid (2,4-D), pH=5.4) to initiate the infection. Callus and Agrobacterium were cocultivated for a period (3 days) (Step 3: cocultivation step). Preferably, callus was cultivated on a solid medium (N6 salt, N6 vitamins, 300 mg/L of casein, 30 g/L of sucrose, 10 g/L of glucose, 40 mg/L of Acetosyringone (AS), 2 mg/L of 2,4-dichlorophenoxyacetic acid (2,4-D) and 3 g/L of plant gelatum, pH=5.8) after the infection step. After this cocultivation step, a “recovery” step can be proceeded. In the “recovery” step, the recovery medium (N6 salt, N6 vitamins, 300 mg/L of casein, 30 g/L of sucrose, 10 g/L of glucose, 2 mg/L of 2,4-dichlorophenoxyacetic acid (2,4-D) and 3 g/L of plant gelatum, pH=5.8) contains at least one kind of known Agrobacterium-inhibiting antibiotics (cephamycin) without the selective agent for plant transfectants (Step 4: recovery step). Preferably, the callus was cultivated on a solid medium culture containing antibiotics but without selective agent so as to eliminate Agrobacterium and to provide a recovery period for the infected cells. Then the inoculated callus was cultivated on a medium containing selective agent (mannose) and the transfected callus was selected (Step 5: selection step). Preferably, the callus was cultivated on a selective solid medium containing selective agent (N6 salt, N6 vitamins, 300 mg/L of casein, 10 g/L of sucrose, 10 g/L of mannose, 2 mg/L of 2,4-dichlorophenoxyacetic acid (2,4-D) and 3 g/L of plant gelatum, pH=5.8), resulting the selective growth of the transfected cells. Then, callus regenerated into plants (Step 6: regeneration step). Preferably, the callus was cultivated on a solid medium containing selective agent (N6 differentiation medium and MS rooting medium) to regenerate into plants.

The obtained resistant callus was transferred to the N6 differentiation medium (N6 salt, N6 vitamins, 300 mg/L of casein, 20 g/L of sucrose, 2 mg/L of 6-benzyladenine, 1 mg/L of naphthylacetic acid and 3 g/L of plant gelatum, pH=5.8) and cultivated and differentiated at 25 T. The differentiated seedlings were transferred to the MS rooting medium (MS salt, MS vitamins, 300 mg/L of casein, 15 g/L of sucrose, 3 g/L of plant gelatum, pH=5.8) and cultivated to about 10 cm in height at 25 T. Next, the seedlings were transferred to and cultivated in the greenhouse until fructification. In the greenhouse, the rice plants were cultivated at 30° C. every day.

2. Verification of Transgenic Rice Plants with Inserted Cry1F Gene Using TaqMan Technique

100 mg of leaves from every transfected rice plant (rice plants transfected with Cry1Fa-01 nucleotide sequence, Cry1Fa-01-Cry1Ab nucleotide sequence and Cry1Fa-02-Vip3A nucleotide sequence, respectively) was taken as sample respectively. Genomic DNA thereof was extracted using DNeasy Plant Maxi Kit (Qiagen) and the copy numbers of Cry1F gene, Cry1Ab gene and Vip3A gene were quantified through Taqman probe-based fluorescence quantitative PCR assay. Wild type rice plant was taken as a control and analyzed according to the processes as described above. Experiments were carried out in triplicate and the results were the mean values.

The specific method for detecting the copy numbers of Cry1F gene, Cry1Ab gene and Vip3A gene was described as follows.

Step 21: 100 mg of leaves from every transfected rice plant (rice plants transfected with nucleotide sequence of Cry1Fa-01, Cry1Fa-01-Cry1Ab or Cry1Fa-02-Vip3A, respectively) was taken and grinded into homogenate in a mortar in liquid nitrogen respectively. It was in triplicate for each sample.

Step 22: the genomic DNAs of the samples above were extracted using DNeasy Plant Mini Kit (Qiagen) following the product instruction thereof.

Step 23: the genome DNA concentrations of the above samples were determined using NanoDrop 2000 (Thermo Scientific).

Step 24: the genome DNA concentrations were adjusted to the same range of 80-100 ng/1.11.

Step 25: the copy numbers of the samples were quantified using Taqman probe-based fluorescence quantitative PCR assay, the quantified sample with known copy number was taken as a standard sample and the wild type rice plant was taken as control. It was carried out in triplicate for every sample and the results were the mean values. Primers and the probes used in the fluorescence quantitative PCR were shown as below.

The following primers and probe were used to detect Cry1Fa-01 nucleotide sequence:

The following primers and probe were used to detect Cry1Fa-01 nucleotide sequence:

Primer 1 (CF1): CAGTCAGGAACTACAGTTGTAAGAGGG (as shown in SEQ ID NO: 10 in the sequence listing);
Primer 2 (CR1): ACGCGAATGGTCCTCCACTAG (as shown in SEQ ID NO: 11 in the sequence listing);
Probe 1 (CP1): CGTCGAAGAATGTCTCCTCCCGTGAAC (as shown in SEQ ID NO: 12 in the sequence listing)

The following primers and probe were used to detect Cry1Ab nucleotide sequence:

Primer 3 (CF2): TGGTGGAGAACGCATTGAAAC (as shown in SEQ ID NO: 13 in the sequence listing);
Primer 4 (CR2): GCTGAGCAGAAACTGTGTCAAGG (as shown in SEQ ID NO: 14 in the sequence listing);
Probe 2 (CP2): CGGTTACACTCCCATCGACATCTCCTTG (as shown in SEQ ID NO: 15 in the sequence listing);

The following primers and probe were used to detect Cry1Fa-02 nucleotide sequence:

Primer 5 (CF3): CAGTCAGGAACTACAGTTGTAAGAGGG (as shown in SEQ ID NO: 16 in the sequence listing);
Primer 6 (CR3): ACGCGAATGGTCCTCCACTAG (as shown in SEQ ID NO: 17 in the sequence listing);
Probe 3 (CP3): CGTCGAAGAATGTCTCCTCCCGTGAAC (as shown in SEQ ID NO: 18 in the sequence listing)

The following primers and probe were used to detect Vip3A nucleotide sequence:

Primers 7 (CF4): ATTCTCGAAATCTCCCCTAGCG (as shown in SEQ ID NO: 19 in the sequence listing);
Primer 8 (CR4): GCTGCCAGTGGATGTCCAG (as shown in SEQ ID NO: 20 in the sequence listing);
Probe 4 (CP4): CTCCTGAGCCCCGAGCTGATTAACACC (as shown in SEQ ID NO: 21 in the sequence listing)

PCR reaction system was as follows:

| | |
|---|---|
| JumpStart ™ Taq ReadyMix ™ (Sigma) | 10 μl |
| 50Xprimer/probe mixture | 1 μl |
| Genomic DNA | 3 μl |
| Water (ddH₂O) | 6 μl |

The 50× primer/probe mixture contained 45 μl of each primer (1 mM), 50 μl of probe (100 μM) and 860 μl of 1×TE buffer and was stored in an amber tube at 4° C.

PCR reaction conditions were provided as follows:

| Step | Temperature | Time |
|---|---|---|
| 21 | 95° C. | 5 min |
| 22 | 95° C. | 30 s |
| 23 | 60° C. | 1 min |

| | |
|---|---|
| 24 | back to step 22 and repeated 40 times |

Data were analyzed using software SDS 2.3 (Applied Biosystems).

The experimental results showed that all the nucleotide sequences of Cry1Fa-01, Cry1Fa-01-Cry1Ab and Cry1Fa-02-Vip3A have been integrated into the genomes of the detected rice plants, respectively. Furthermore, rice plants transfected with nucleotide sequences of Cry1Fa-01, Cry1Fa-01-Cry1Ab and Cry1Fa-02-Vip3A respectively contained single copy of Cry1F gene, Cry1Ab gene, and/or Vip3A gene respectively.

### Example 6

### Detection of Pesticidal Protein in Transgenic Rice Plants

### 1. Content Detection of the Pesticidal Protein in Transgenic Rice Plants

Solutions involved in this experiment were as follows:

Extraction buffer: 8 g/L of NaCl, 0.2 g/L of KH₂PO₄, 2.9 g/L of Na₂HPO₄.12H₂O, 0.2 g/L of KCl, 5.5 ml/L of Tween-20, pH=7.4;
Washing buffer PBST: 8 g/L of NaCl, 0.2 g/L of KH₂PO₄, 2.9 g/L of Na₂HPO₄.12H₂O, 0.2 g/L of KCl, 0.5 ml/L of Tween-20, pH=7.4;
Stop solution: 1 M HCl.

3 mg of fresh leaves from each transfected rice plant (rice plant transfected with nucleotide sequence of Cry1Fa-01, Cry1Fa-01-Cry1Ab or Cry1Fa-02-Vip3A, respectively) was taken as a sample respectively. All the samples were grinded in liquid nitrogen and 800 μl of the extraction solution was added therein. The mixture was centrifuged at 4000 rpm for 10 min and the supernatant was diluted 40 folds with the extraction buffer and 80 μl of the diluted supernatant was taken out for an ELISA test. The ratio of pesticidal protein (Cry1Fa protein, Cry1Ab protein and Vip3A protein)/fresh weight of leaves was determined using an ELISA (enzyme-linked immunosorbent assay) kit (ENVIRLOGIX Co., Cry1Fa kit, Cry1Ab kit and Vip3A kit) and the specific method was shown in the product instruction.

At the same time, the wild type rice plants and rice plants identified as non-transgenic with the Taqman technique were taken as controls and analyzed following the above methods.

There were three strains (S10, S11, and S12) containing the inserted nucleotide sequence Cry1Fa-01, three strains (S13, S14 and S15) containing the inserted nucleotide sequence Cry1F-01-Cry1Ab and three strains (S16, S17 and S18) containing the inserted nucleotide sequence Cry1Fa-02-Vip3A. There presented one strain identified as non-transgenic (NGM2) via Taqman technique and one wild type strain (CK2). Three plants of each strain were selected for further tests and each plant was repeated 6 times.

Pesticidal protein (Cry1Fa protein) contents in the transgenic rice plants were shown in Table 6. Pesticidal protein (Cry1Ab protein) contents in the transgenic rice plants were shown in Table 7. Pesticidal protein (Vip3A protein) contents in the transgenic rice plants were shown in Table 8. Ratios (ng/g) of the average expression is value of the pesticidal protein (Cry1Fa protein) vs fresh weight of the leaves of the rice plants containing nucleotide sequence of Cry1Fa-01, Cry1Fa-01-Cry1Ab or Cry1Fa-02-Vip3A were 4194.80, 4140.16 or 4227.60 respectively. Ratio (ng/g) of the average expression value of the pesticidal protein (Cry1Ab protein) vs fresh weight of the leaves of the rice plant containing nucleotide sequence Cry1Fa-01-Cry1Ab was 13861.64. Ratio (ng/g) of the average expression value of the pesticidal protein (Vip3A protein) vs fresh weight of the leaves of the rice plant containing nucleotide sequence Cry1Fa-02-Vip3A was 3913.97. These results showed that all Cry1Fa protein, Cry1Ab protein and Vip3A protein were expressed highly and stably in rice plant.

TABLE 6
Average expression values of Cry1Fa protein in transgenic rice plants

| | The amount of | | | |
|---|---|---|---|---|
| | Cry1Fa protein | | | |
| | in each plant (ng/g) | | | The amount values of |
| | (repeated 6 times | | | Cry1Fa protein expressed |
| | for each plant) | | | in each line (ng/g) |
| line | 1 | 2 | 3 | Average expression value (ng/g) |
| S10 | 4922.79 | 4845.05 | 3420.91 | 4194.80 |
| S11 | 4769.75 | 4316.96 | 3676.25 | |
| S12 | 3876.94 | 4665.52 | 3259.06 | |
| S13 | 4019.57 | 3762.15 | 3958.23 | 4140.16 |
| S14 | 4586.27 | 4585.64 | 4158.94 | |
| S15 | 4035.26 | 4062.15 | 4093.26 | |
| S16 | 4502.02 | 4973.23 | 3278.55 | 4227.60 |
| S17 | 3938.5 | 4266.58 | 4278.23 | |
| S18 | 3664.84 | 4897.37 | 4249.15 | |
| NGM2 | −2.36 | 0 | −3.54 | 0 |
| CK2 | 0 | −0.14 | −5.18 | 0 |

TABLE 7
Average expression values of Cry1Ab protein in transgenic rice plants

| | Expression values | | | Expression values |
|---|---|---|---|---|
| | of Cry1Ab protein | | | of Cry1Ab |
| | in a single plant (ng/g) | | | protein in each |
| | (repeated 6 times | | | strain (ng/g) |
| | for each plant) | | | Average expression |
| Strain | 1 | 2 | 3 | value (ng/g) |
| S13 | 12359.18 | 15500.82 | 12940.71 | 13861.64 |
| S14 | 14465.69 | 13589.34 | 13876.85 | |
| S15 | 12367.59 | 13678.21 | 15976.34 | |
| NGM2 | −3.58 | 0 | −2.45 | 0 |
| CK2 | 0 | −0.78 | −5.41 | 0 |

TABLE 8
Average expression values of Vip3A protein in transgenic rice plants

| | Expression values | | | |
|---|---|---|---|---|
| | of Vip3A protein in | | | |
| | a single plant | | | |
| | (ng/g) (repeated | | | Expression values of Vip3A |
| | 6 times | | | protein in each |
| | for each plant) | | | strain (ng/g) Average |
| Strain | 1 | 2 | 3 | expression values (ng/g) |
| S16 | 3921.15 | 3769.52 | 4016.86 | 3913.97 |
| S17 | 3797.35 | 3684.75 | 3926.49 | |
| S18 | 4035.16 | 3906.52 | 4167.95 | |
| NGM2 | −2.64 | 0 | −5.51 | 0 |
| CK2 | 0 | −0.89 | −9.31 | 0 |

### 2. Insect-Resistance Effect Test of the Transgenic Rice Plants

Sesamia inferen-resistance effects of the rice plants transfected with Cry1Fa-01 nucleotide sequence, rice plants transfected with Cry1Fa-01-Cry1Ab nucleotide sequence, rice plants transfected with Cry1Fa-02-Vip3A nucleotide sequence, the wild type rice plants and the rice plants identified as non-transgenic with Taqman technique were tested.

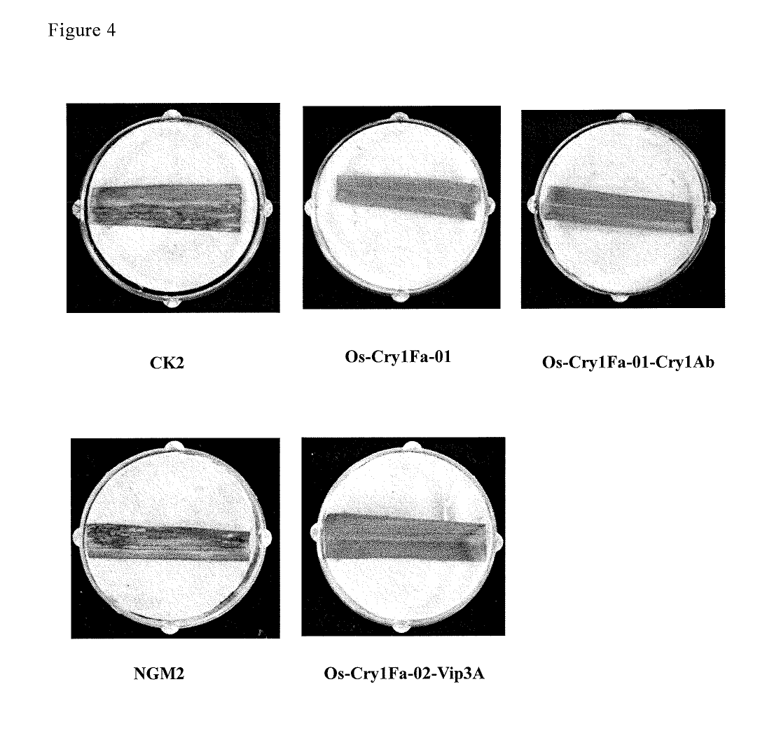

Fresh leaves of the rice plants transfected with Cry1Fa-01 nucleotide sequence, Cry1Fa-01-Cry1Ab nucleotide sequence or Cry1Fa-02-Vip3A nucleotide sequence, the wild type rice plant and rice plant identified as non-transgenic with Taqman technique (tillering stage) were taken respectively and washed with sterile water, and the water remained on the leaf surfaces were dried with a piece of gauze. The leaf veins were removed and at the same time the leaves were cut into long strips (1 cm*3 cm). One strip was put on a filter paper on the bottom of a round plastic Petri dish. The filter paper was wet with distilled water and 10 artificially fed Sesamia inferens (newly hatched larvae) were put in each round plastic Petri dish. Then the Petri dish was covered and kept for 3 days in a condition with a temperature of 26-28° C., relative humidity 70%-80%, photoperiod (light/dark)16:8. Then, statistics of leaf feeding, larvae survival and development conditions were carried out, and average corrected mortality and larvae weight from every sample were calculated. Average corrected mortality M=(Mt−Mc)/(1−Mc)*100%, wherein M is average corrected mortality (%), Mt is the average mortality (%) of the insects on rice plants to be tested, Mc is the average mortality (%) of the insects on control plants (CK2). The insect-resistance grading standard was shown in Table 4. Three strains (S10, S11, and S12) of rice plants transfected with Cry1Fa-01 nucleotide sequence; three strains (S13, S14, and S15) of rice plants transfected with Cry1F-01-Cry1Ab nucleotide sequence; three strains (S16, S17, and S18) of rice plants transfected with Cry1Fa-02-Vip3A nucleotide sequence; one strain identified as non-transgenic (NGM2) via Taqman technique and one wild type strain (CK2) were selected. Three plants of each strain were tested and each plant is repeated 6 times. The results were shown in Table 9 and FIG. 4.

TABLE 9
Insect-resistances of the transgenic rice plants inoculated
with Sesamia inferens

| | | | Total | | | | |
|---|---|---|---|---|---|---|---|
| | | | weight of | | | | |
| | Larvae numbers | | the | | | | |
| | Inoc- | Sur- | survived | Corrected | | Weight/each | |
| | ulated | vived | larvae | mortality | | insect | |
| | larvae | larvae | (mg) | (%) | Average | (mg) | Average |
| S10-1 | 10 | 1 | 0.2 | 88.9 | | 0.20 | |
| S10-2 | 10 | 1 | 0.1 | 88.9 | | 0.10 | |
| S10-3 | 10 | 0 | 0 | 100.0 | | 0 | |
| S11-1 | 10 | 2 | 0.4 | 77.8 | | 0.20 | |
| S11-2 | 10 | 1 | 0.1 | 88.9 | | 0.10 | |
| S11-3 | 10 | 0 | 0 | 100.0 | | 0 | |
| S12-1 | 10 | 0 | 0 | 100.0 | | 0 | |
| S12-2 | 10 | 0 | 0 | 100.0 | | 0 | |
| S12-3 | 10 | 1 | 0.1 | 88.9 | | 0.10 | |
| S13-1 | 10 | 1 | 0.1 | 88.9 | 90.1 | 0.10 | 0.13 |
| S13-2 | 10 | 2 | 0.3 | 77.8 | | 0.15 | |
| S13-3 | 10 | 0 | 0 | 100 | | 0 | |
| S14-1 | 10 | 2 | 0.3 | 77.8 | | 0.20 | |
| S14-2 | 10 | 0 | 0 | 100 | | 0 | |
| S14-3 | 10 | 1 | 0.1 | 88.9 | | 0.20 | |
| S15-1 | 10 | 1 | 0.1 | 88.9 | | 0.10 | |
| S15-2 | 10 | 1 | 0.2 | 88.9 | | 0.20 | |
| S15-3 | 10 | 0 | 0 | 100 | | 0 | |
| S16-1 | 10 | 2 | 0.4 | 77.8 | 92.6 | 0.20 | 0.13 |
| S16-2 | 10 | 1 | 0.1 | 88.9 | | 0.10 | |
| S16-3 | 10 | 0 | 0 | 100 | | 0 | |
| S17-1 | 10 | 0 | 0 | 100 | | 0 | |
| S17-2 | 10 | 0 | 0 | 100 | | 0 | |
| S17-3 | 10 | 1 | 0.1 | 88.9 | | 0.10 | |
| S18-1 | 10 | 2 | 0.2 | 77.8 | | 0.10 | |
| S18-2 | 10 | 0 | 0 | 100 | | 0 | |
| S18-3 | 10 | 0 | 0 | 100 | | 0 | |
| NGM2- | 10 | 8 | 142.2 | 11.1 | 3.7 | 17.78 | 14.29 |
| 1 | | | | | | | |
| NGM2- | 10 | 9 | 105.3 | 0 | | 11.70 | |
| 2 | | | | | | | |
| NGM2- | 10 | 9 | 120.4 | 0 | | 13.38 | |
| 3 | | | | | | | |
| CK2-1 | 10 | 9 | 123.5 | | 0 | 13.72 | 15.20 |
| CK2-2 | 10 | 9 | 125.6 | | | 13.96 | |
| CK2-3 | 10 | 9 | 161.3 | | | 17.92 | |

Results of Table 9 and FIG. 4 showed that average corrected mortalities of most rice plants transfected with the Cry1Fa-01 nucleotide sequence, rice plants transfected with the Cry1Fa-01-Cry1Ab and rice plants transfected with the Cry1Fa-02-Vip3A were around or above 90%, and average corrected mortalities of some strains were up to 100%. Compared with this, the average corrected mortalities of wild type rice plants were generally round or below 10%. Compared with the wild type rice plants, control efficiencies against newly hatched larvae of rice plants transfected with the Cry1Fa-01 nucleotide sequence, rice plants transfected with the Cry1Fa-01-Cry1Ab and rice plants transfected with the Cry1Fa-02-Vip3A were almost 100% and the individual larvae scarcely survived also substantially stopped development. Furthermore, rice plants transfected with the Cry1Fa-01 nucleotide sequence, rice plants transfected with the Cry1Fa-01-Cry1Ab and rice plants transfected with the Cry1Fa-02-Vip3A were only slightly harmed in general.

It was thereby demonstrated that all rice plants transfected with the Cry1Fa-01 nucleotide sequence, rice plants transfected with the Cry1Fa-01-Cry1Ab and rice plants transfected with the Cry1Fa-02-Vip3A showed high Sesamia inferen-resistant activity, which was enough to result in a harmful effect to the growth of Sesamia inferen and to control Sesamia inferen.

The above experimental results also showed that Sesamia inferen control of corn plants transfected with the Cry1Fa-01 nucleotide sequence, corn plants transfected with the Cry1Fa-01-Cry1Ab, corn plants transfected with the Cry1Fa-02-Vip3A, rice plants transfected with the Cry1Fa-01 nucleotide sequence, rice plants transfected with the Cry1Fa-01-Cry1Ab and rice plants transfected with the Cry1Fa-02-Vip3A was due to the Cry1F proteins expressed in these plants themselves. Therefore, as well-known by one skilled in the art, based on the same toxic action of Cry1F proteins to Sesamia inferen, other similar transgenic plants capable of expressing Cry1F proteins can be obtained so as to control Sesamia inferen. Cry1F proteins in this invention included but were not limited to those whose amino acid sequences were provided in the specific embodiments of present invention. At the same time, these transgenic plants can also produce at least one second pesticidal protein different from Cry1F protein such as Cry1Ab protein, Cry1Ac protein, Cry1Ba protein or Vip3A protein, etc.

In conclusion, the methods for controlling pest in the present invention were to control Sesamia inferen pest with Cry1F protein produced in the plants, which can kill Sesamia inferens. Compared with the agricultural control, chemical control and biological control currently used in the prior art, the present invention can protect the whole plant during whole growth period from the harm of Sesamia inferen. Furthermore, it causes no pollution and no residue and provides a stable and thorough control effect. Also it is simple, convenient and economic.

Finally what should be explained is that all the above examples are merely intentioned to illustrate the technical solutions of present invention rather than to restrict present invention. Although detailed description of this invention has been provided by referring to the preferable examples, one skilled in the art should understand that the technical solutions of the invention can be modified or equivalently substituted while still fall within the spirit and scope of the present invention.

What is claimed is:

1. A method for controlling Sesamia inferens comprising a step of contacting Sesamia inferens with Cry1F protein.

2. The method of claim 1, wherein the Cry1F protein is Cry1Fa protein.

3. The method of claim 2, wherein the Cry1Fa protein is present in a plant cell that can express the Cry1Fa protein, and Sesamia inferens contacts with the Cry1Fa protein by ingestion of the cell.

4. The method of claim 3, wherein the Cry1Fa protein is present in a transgenic plant that expresses the Cry1Fa protein, and Sesamia inferens contacts with the Cry1Fa protein by ingestion of a tissue of the transgenic plant such that the growth of Sesamia inferens is suppressed or even resulting in the death of Sesamia inferens to achieve the control of the damage caused by Sesamia inferens.

5. The method of claim 4, wherein the transgenic plant is in any growth period.

6. The method of claim 4, wherein the tissue of the transgenic plants is selected from the group consisting of lamina, stalk, tassel, ear, anther and filament.

7. The method of claim 4, wherein the control of the damage caused by Sesamia inferens to the plant is independent of the planting location.

8. The method of claim 4, wherein the control of the damage caused by Sesamia inferens to the plant is independent of the planting time.

9. The method of claim 4, wherein the plant is selected from the group consisting of corn, rice, sorghum, wheat, millet, cotton, reed, sugarcane, water bamboo, broad bean and rape.

10. The method of claim 3, wherein prior to the step of contacting, a step of growing a plant which contains a polynucleotide encoding the Cry1Fa protein is performed.

11. The method of claim 2, wherein the amino acid sequence of the Cry1Fa protein comprises the amino acid sequence of SEQ ID NO: 1 or SEQ ID NO: 2.

12. The method of claim 11, wherein the nucleotide sequence encoding Cry1Fa protein comprises a nucleotide sequence of SEQ ID NO: 3 or SEQ ID NO: 4.

13. The method of claim 3, wherein the plant further contains at least a second nucleotide sequence, which is different from that encoding the Cry1Fa protein.

14. The method of claim 13, wherein the second nucleotide encodes a Cry-like pesticidal protein, a Vip-like pesticidal protein, a protease inhibitor, lectin, α-amylase or peroxidase.

15. The method of claim 14, wherein the second nucleotide encodes Cry1Ab protein, Cry1Ac protein, Cry1Ba protein or Vip3A protein.

16. The method of claim 15, wherein the second nucleotide comprises a nucleotide sequence of SEQ ID NO: 5 or SEQ ID NO: 6.

17. The method of claim 13, wherein the second nucleotide is dsRNA which inhibits important gene(s) of a target pest.


SEQUENCE LISTING (21 sequences)

SEQ ID NO: 1 - protein, 605 aa, Artificial Sequence
  amino acid sequence of Cry1Fa-01
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLE

SEQ ID NO: 2 - protein, 1148 aa, Artificial Sequence
  amino acid sequence of Cry1Fa-02
MENNIQNQRVPYNCPNNPEVEILNEERSTGRLPLDISLSLTRFLLSEFVPGVGVAFGLFDLIWGFITPSDWSLFLLQIEQLIEQRIETLERNRAITTLRGLADSYETYIEALREREANPNNAQPREDVRIRFANTDDALITATNNFTLTSFETPLLSVYVQAANLHLSLLRDAVSFGQGWGLDIATANNHYNRLINLIHRYTKHCLDTYNQGLENLRGTNTRQWARFNQFRRDLTLTVLDTVALFPNYDVRTYPTQTSSQLTREIYTSSVIEDSPVSANIPNGFNRAEFGARPPHLTDFMNSLFVTAETVRSQTVRGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQTPLVKAHTLQSGTTVVRGPGFTGGDILRRTSGGPFAYTIVNINGQLPQRYRARIRHASTTNLRIYVTVAGERIFAGQFNKTMDTGDPLTFQSFSYATINTAFTFPMSQSSFTVGADTFSSGNEVYIDRFELIPVTATLEAESDLERAQKAVNALFTSSNQIGLKTDVTDYHIDRVSNLVECLSDEFCLDEKKELSEKVKHAKRLSDERNLLQDPNFRGINRQLDRGWRGSTDTTIQGGDDAFKENYVTLLGTSDERYPTYLYQKIDESKLKAYTRYQLRGYIEDSQDLEIYLIRYNAKHETVNVPGTGSLWPLSAPSPIGKCAHHSHHFSSDIDVGCTDLNEDLGVWAIFKIKTQDGHARLGNLEFLEEKPLVGEALARVKRAEKKWRDKREKLEWETNTVYKEAKESVDALFVNSQYDRLQADTNIAMIHAADKRVHSIREAYLPELSVIPGVNAAIFEELEGRIFTAPSLYDARNVIKNGDFNNGLSCWNVKGHVDVEEQNNHRSVPVVPEWEAEVSQEVRACPGRGYTLRVTAYKEGYGEGCVTIHEIENNTDELKFSNCVEEEVYPNNTVTCNDYTATQEEHEGTYTSRNRGYDGAYESNSSAPADYASAYEEKAYTDGRRDNPCEPNRGYGDYTPLPAGYVTKELEHLPETDKVWIEIGETEGTLIVDSVELPLMEE

SEQ ID NO: 3 - DNA, 1818 bp, Artificial Sequence
  nucleotide sequence encoding Cry1Fa-01
atggagaacaacatacagaatcagtgcgtcccctacaactgcctcaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagttgattgaacaaaggattgagaccttggaaaggaatcgggccatcactacccttcgtggcttagcagacagctatgagatctacattgaagcactaagagagtgggaagccaatcctaacaatgcccaactgagagaagatgtgcgtatacgctttgctaacacagatgatgctttgatcacagccatcaacaacttcacccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctgagtttggagtcagaccaccccatctcatggacttcatgaactctttgtttgtgactgcagagactgttagatcccaaactgtgtggggaggacacttagttagctcacgcaacacggctggcaatcgtatcaactttcctagttacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagattcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgctatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgagtaa

SEQ ID NO: 4 - DNA, 3447 bp, Artificial Sequence
  nucleotide sequence encoding Cry1Fa-02
atggagaacaacatacagaatcagcgcgtcccctacaactgccccaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagctgattgaacaaaggattgagaccttggaaaggaatcgggccatcactacccttcgtggcttagcagacagctatgagacctacattgaagcactaagagagcgggaagccaatcctaacaatgcccaaccgagagaagatgtgcgtatacgctttgctaacacagatgatgctttgatcacagccaccaacaacttcacccttaccagcttcgagacccctcttctctcggtctatgttcaagctgccaacctgcacttgtcactactgcgcgacgctgtgtcgtttgggcaaggttggggactggacatagctactgccaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacacagttgctctctttccgaactacgatgttcgtacctatccgactcaaacgtcatcccaacttacaagggagatctacaccagttcagtcattgaagactctccagtttctgcgaacatacccaatggtttcaacagggctgagtttggagccagaccaccccatctcacggacttcatgaactctttgtttgtgactgcagagactgttagatcccaaactgtgcggggaggacacttagttagctcacgcaacacggctggcaatcgtatcaactttcctagctacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagactcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgccatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgaggcagagtctgacttggaaagagcacagaaggcggtgaatgctctgttcacttcgtccaatcagattgggctcaagacagatgtgactgactatcacatcgatcgcgtttccaaccttgttgagtgcctctctgatgagttctgtttggatgagaagaaggagttgtccgagaaggtcaaacatgctaagcgacttagtgatgagcggaacttgcttcaagatcccaactttcgcgggatcaacaggcaactagaccgtggatggaggggaagtacggacaccaccattcaaggaggtgatgatgcgttcaaggagaactatgtcacgctcttgggtacctctgacgagcgctatccaacatacctgtaccagaagatagatgaatcgaaactcaaagcctacacaagataccagttgagaggttacatcgaggacagtcaagaccttgagatctacctcatcagatacaacgccaaacatgagacagtcaatgtgcctgggacgggttcactctggccactttcagccccaagtcccatcggcaagtgcgcccaccactcacaccacttctcctcggacatagacgttggctgtaccgacctgaacgaagacctcggtgtgtgggcgatcttcaagatcaagactcaagatggccatgccaggctaggcaatctggagttcctagaagagaaaccacttgttggagaagccctcgctagagtgaagagggctgagaagaagtggagggacaagagagagaagttggaatgggaaacaaacactgtgtacaaagaagccaaagaaagcgttgacgctctgtttgtgaactcccagtatgataggctccaagctgataccaacatagctatgattcatgctgcagacaaacgcgttcatagcattcgggaagcttaccttcctgaacttagcgtgattccgggtgtcaatgctgctatctttgaagagttagaagggcgcatcttcactgcaccctccttgtatgatgcgaggaatgtcatcaagaatggtgacttcaacaatggcctatcctgctggaatgtgaaagggcacgtagatgtagaagaacagaacaatcaccgctctgtccctgttgttcctgagtgggaagcagaagtttcacaagaagttcgtgcctgtcccggccgtggctacactcttcgtgttaccgcgtacaaagaaggatacggagaaggttgcgtcaccatacacgagattgagaacaacaccgacgagctgaagttcagcaactgcgtcgaggaggaagtctacccaaacaacaccgtaacttgcaatgactacactgcgactcaagaggagcacgagggtacttacacttctcgcaatcgaggatacgatggagcctatgagagcaactcttctgcacccgctgactatgcatcagcctacgaggagaaggcttacaccgatggacgtagggacaacccttgcgaacctaacagaggctacggggactacacaccgttaccagccggctatgtcaccaaagagctagagcaccttccagaaaccgacaaggtttggattgagattggagaaacggaaggaacactcattgttgatagcgtggagttacctctgatggaggaataa

SEQ ID NO: 5 - DNA, 1848 bp, Artificial Sequence
  nucleotide sequence encoding Cry1Ab
atggacaacaacccaaacatcaacgaatgcattccatacaactgcttgagtaacccagaagttgaagtacttggtggagaacgcattgaaaccggttacactcccatcgacatctccttgtccttgacacagtttctgctcagcgagttcgtgccaggtgctgggttcgttctcggactagttgacatcatctggggtatctttggtccatctcaatgggatgcattcctggtgcaaattgagcagttgatcaaccagaggatcgaagagttcgccaggaaccaggccatctctaggttggaaggattgagcaatctctaccaaatctatgcagagagcttcagagagtgggaagccgatcctactaacccagctctccgcgaggaaatgcgtattcaattcaacgacatgaacagcgccttgaccacagctatcccattgttcgcagtccagaactaccaagttcctctcttgtccgtgtacgttcaagcagctaatcttcacctcagcgtgcttcgagacgttagcgtgtttgggcaaaggtggggattcgatgctgcaaccatcaatagccgttacaacgaccttactaggctgattggaaactacaccgaccacgctgttcgttggtacaacactggcttggagcgtgtctggggtcctgattctagagattggattagatacaaccagttcaggagagaattgaccctcacagttttggacattgtgtctctcttcccgaactatgactccagaacctaccctatccgtacagtgtcccaacttaccagagaaatctatactaacccagttcttgagaacttcgacggtagcttccgtggttctgcccaaggtatcgaaggctccatcaggagcccacacttgatggacatcttgaacagcataactatctacaccgatgctcacagaggagagtattactggtctggacaccagatcatggcctctccagttggattcagcgggcccgagtttacctttcctctctatggaactatgggaaacgccgctccacaacaacgtatcgttgctcaactaggtcagggtgtctacagaaccttgtcttccaccttgtacagaagacccttcaatatcggtatcaacaaccagcaactttccgttcttgacggaacagagttcgcctatggaacctcttctaacttgccatccgctgtttacagaaagagcggaaccgttgattccttggacgaaatcccaccacagaacaacaatgtgccacccaggcaaggattctcccacaggttgagccacgtgtccatgttccgttccggattcagcaacagttccgtgagcatcatcagagctcctatgttctcatggattcatcgtagtgctgagttcaacaatatcattccttcctctcaaatcacccaaatcccattgaccaagtctactaaccttggatctggaacttctgtcgtgaaaggaccaggcttcacaggaggtgatattcttagaagaacttctcctggccagattagcaccctcagagttaacatcactgcaccactttctcaaagatatcgtgtcaggattcgttacgcatctaccactaacttgcaattccacacctccatcgacggaaggcctatcaatcagggtaacttctccgcaaccatgtcaagcggcagcaacttgcaatccggcagcttcagaaccgtcggtttcactactcctttcaacttctctaacggatcaagcgttttcacccttagcgctcatgtgttcaattctggcaatgaagtgtacattgaccgtattgagtttgtgcctgccgaagttaccttcgaggctgagtactga

SEQ ID NO: 6 - DNA, 2370 bp, Artificial Sequence
  nucleotide sequence encoding Vip3A
atgaacaagaacaacaccaagctctccacacgggcacttccctcctttattgactactttaatggcatctatgggtttgctacggggatcaaggacattatgaacatgatcttcaagacagacactggcggggatcttacgctcgacgagattcttaagaatcagcaactcctgaacgatatctctggcaagctggacggcgtgaatgggtcacttaacgacctcatcgctcaggggaatctcaacacagaactgtctaaggagatcctcaagattgcaaatgagcagaaccaagttcttaatgatgtgaacaataagctcgacgccatcaacacaatgcttcgcgtgtacctcccaaagattactagcatgctctcggacgtcatgaagcagaactacgcgctgtcccttcaaattgagtatctgagcaagcagcttcaagaaatctcggacaagctggatatcattaatgtgaacgtcctcatcaacagcaccctgacggagattacaccggcgtaccagaggatcaagtatgtgaatgagaagttcgaggaactcacttttgctacagaaacttccagcaaggtcaagaaggatggctcaccagccgacatcctggatgagcttacagaactcactgagctggcgaagtccgtgaccaagaatgacgtcgatggcttcgagttttacctgaacacgttccacgacgttatggtgggcaacaatctttttgggcggagcgctctcaagactgcatcggaactgatcaccaaggagaacgttaagacgagcggctcggaggtcgggaatgtttacaacttccttatcgtcctcaccgcactccaggcccaagcgtttctcacgctgaccacctgccgcaagctcctcggcctcgcagacatcgattacacctccatcatgaacgagcacctgaacaaggagaaggaggagttccgcgtgaatatccttccgacactctcgaacactttttctaatccaaactacgctaaggtcaagggctccgacgaagatgcaaagatgatcgttgaggccaagcctggccatgcgctcatcgggttcgagatttctaacgactcaattaccgtgctgaaggtctacgaggcgaagctcaagcagaattatcaagtggacaaggattctctgtcagaggttatctacggcgacatggataagctgctttgccctgatcagtccgagcaaatctactatacgaacaatattgtcttccccaacgaatacgtgatcaccaagattgactttacgaagaagatgaagacactccggtacgaggtgacggctaacttctatgattcgtctacgggcgagatcgacctcaacaagaagaaggtcgaatcatccgaggccgaatacagaaccctgtcggcgaacgacgatggcgtgtatatgcctcttggggtcatttctgagaccttcctcacgcccatcaatggctttgggctccaggcagatgagaactcccgcctgatcacccttacgtgcaagagctacctcagggagctgctgcttgccaccgacctctctaacaaggaaacgaagctgatcgttccgccatcaggcttcatctccaatattgtggagaacgggtcaattgaggaagataatctggaaccgtggaaggctaacaataagaacgcatacgttgaccacacaggcggggtgaatggcactaaggcgctctatgtgcataaggatggtggcatctcccagttcattggcgacaagctgaagccgaagacagaatacgtgattcaatatactgtgaagggcaagccaagcatccacctcaaggatgagaacacagggtacatccattacgaagatactaacaacaacctggaggactaccagacaatcaataagaggttcacaactggcactgacctgaagggggtctatcttattctcaagtcccagaatggcgatgaggcctggggcgacaacttcatcattctcgaaatctcccctagcgagaagctcctgagccccgagctgattaacaccaataactggacatccactggcagcacgaatatctcggggaacaccctgacgctttaccagggcgggagaggcattctgaagcagaacctccaactggattcgttctctacctacagagtctatttttcagtttccggcgacgcgaatgtgcgcatcaggaactcgcgggaagtcctcttcgagaagagatacatgtctggcgctaaggatgtgtcagaaatgttcaccacgaagtttgagaaggacaacttttatatcgaactgtcccaagggaataacctctacggcggccccattgttcatttttacgacgtgagcatcaagtga

SEQ ID NO: 7 - DNA, 1992 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 8 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 9 - DNA, 1176 bp, Escherichia coli
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 10 - DNA, 27 bp, Artificial Sequence
  Primer 1
cagtcaggaactacagttgtaagaggg

SEQ ID NO: 11 - DNA, 21 bp, Artificial Sequence
  Primer 2
acgcgaatggtcctccactag

SEQ ID NO: 12 - DNA, 27 bp, Artificial Sequence
  Probe 1
cgtcgaagaatgtctcctcccgtgaac

SEQ ID NO: 13 - DNA, 21 bp, Artificial Sequence
  Primer 3
tggtggagaacgcattgaaac

SEQ ID NO: 14 - DNA, 23 bp, Artificial Sequence
  Primer 4
gctgagcagaaactgtgtcaagg

SEQ ID NO: 15 - DNA, 28 bp, Artificial Sequence
  Probe 2
cggttacactcccatcgacatctccttg

SEQ ID NO: 16 - DNA, 27 bp, Artificial Sequence
  Primer 5
cagtcaggaactacagttgtaagaggg

SEQ ID NO: 17 - DNA, 21 bp, Artificial Sequence
  Primer 6
acgcgaatggtcctccactag

SEQ ID NO: 18 - DNA, 27 bp, Artificial Sequence
  Probe 3
cgtcgaagaatgtctcctcccgtgaac

SEQ ID NO: 19 - DNA, 22 bp, Artificial Sequence
  Primer 7
attctcgaaatctcccctagcg

SEQ ID NO: 20 - DNA, 19 bp, Artificial Sequence
  Primer 8
gctgccagtggatgtccag

SEQ ID NO: 21 - DNA, 27 bp, Artificial Sequence
  Probe 4
ctcctgagccccgagctgattaacacc